(19) United States
(12) Patent Application Publication
(10) Pub. No.: US 2022/0054318 A1
(43) Pub. Date: Feb. 24, 2022

(54) WEARABLE SYSTEM AND METHOD FOR MODIFICATION OF FLUID ENVIRONMENT OF AN EAR

(71) Applicant: Giner Life Sciences, Inc., Newton, MA (US)

(72) Inventors: Melissa N. Schwenk, Somerville, MA (US); Ernesto Victor Ruiz, Wharton, NJ (US); Simon G. Stone, Arlington, MA (US); Linda A. Tempelman, Lincoln, MA (US)

(21) Appl. No.: 17/408,396

(22) Filed: Aug. 21, 2021

Related U.S. Application Data
(60) Provisional application No. 63/068,769, filed on Aug. 21, 2020.

(51) Int. Cl. A61F 11/00; A61M 13/00; A61M 31/00

(52) U.S. Cl. CPC A61F 11/006; A61M 13/003; A61M 31/00; A61M 2210/0662; A61M 2205/8206; A61M 2205/502; A61M 2202/0208

(57) ABSTRACT

System and method for modification of fluid environment of an ear. In one embodiment, the system includes an earpiece mountable within an ear canal. The earpiece includes a fluid delivery path for fluid to be delivered to the ear and a fluid removal path for fluid to be removed from the ear. The system also includes an electronics housing. The electronics housing may be directly mounted on the earpiece or positioned outside the ear. The system further includes an electrochemical gas generating device positioned within the electronics housing. In use, oxygen or the like is generated by the electrochemical gas generating device and is conveyed through the fluid delivery path of the earpiece, emerging from the earpiece distal end. The gas released from the earpiece causes fluid in the ear to be swept into the fluid removal path of the earpiece and eventually expelled to the outside of the ear.

### CROSS-REFERENCE TO RELATED APPLICATIONS

The present application claims the benefit under 35 U.S.C. 119(e) of U.S. Provisional Patent Application No. 63/068,769, inventors Melissa N. Schwenk et al., filed Aug. 21, 2020, the disclosure of which is incorporated herein by reference in its entirety.

### FEDERALLY SPONSORED RESEARCH OR DEVELOPMENT

This invention was made with government support under SBIR Phase I NIH-NIDCD Grant Number R43DC017626 entitled “Wearable Ear Oxygenation” awarded by the National Institute of Health National Institute on Deafness and Other Communication Disorders. The government has certain rights in the invention.

### BACKGROUND OF THE INVENTION

The present invention relates generally to methods and systems for modifying the fluid environment of an ear and relates more particularly to a novel method and system for modifying the fluid environment of an ear.

In certain situations, it may be desirable to modify the fluid environment of an ear. For example, it may be desirable to remove excess moisture from the outer auditory canal and/or middle ear of a person, for example, to reduce the occurrence of otitis externa, more commonly known as swimmer's ear. A common technique for drying the ear is to use a hair dryer to blow heated air into the ear. Unfortunately, however, this technique is less than optimal. This is at least for the reason that it is difficult to direct air from a hair dryer into an ear without bringing the hair dryer so close to the ear that the ear is subjected to excessive temperature and/or pressure from the hair dryer.

One approach to addressing the aforementioned problem is disclosed in U.S. Patent Application Publication No. US 2011/0099832 A1, inventor Bikhazi, published May 5, 2011, which is incorporated herein by reference. According to the aforementioned publication, there is provided an ear drying device that includes an adapter sleeve defining an outer surface and an inner surface defining a fluid chamber. A fluid flow is generated through the fluid chamber when the adapter sleeve is engaged with a hair dryer. An exhaust vent extends from the outer surface to the inner surface. An inlet vent extends from the outer surface to the inner surface and is spaced axially from the exhaust vent. The adapter sleeve is sized and configured to draw ambient air through the inlet vent into the fluid chamber in response to fluid flowing through the fluid chamber. A diffuser is disposable within the adapter sleeve and is sized and configured to direct a portion of the fluid flow toward the exhaust vent. An ear piece is engageable with the adapter sleeve and is configured to direct fluid toward the user's ear.

Another approach to addressing the above-described problem is disclosed in PCT International Publication No. WO 2010/124846 A1, published Nov. 4, 2010, which is incorporated herein by reference. According to the aforementioned publication, there is disclosed a device for drying the outer auditory canal and/or the middle ear of a person, wherein the device comprises a housing to be worn behind the ear, a blower integrated into the housing for producing an air stream, and a connecting line connected to the housing and capable of insertion into the outer auditory canal for conducting the air stream from the housing into the outer auditory canal through an outlet opening on the auditory canal side. In an alternate configuration, a housing to be worn in the ear is provided.

One shortcoming that has been identified by the present inventors with the above-described approaches is that there is likely to be an undesirable acoustic impact on the user due to the loud noise made by the hair dryer or blower. Another shortcoming that has been identified by the present inventors with the above-described approaches is that these approaches are limited to supplying the ear with ambient air or heated ambient air.

Other documents that may be of interest may include the following, both of which are incorporated herein by reference: PCT International Publication No. WO 2004/030589 A1, published Apr. 15, 2004; and European Patent Application Publication No. EP 0 937 422 A2, published Aug. 25, 1999.

### SUMMARY OF THE INVENTION

It is an object of the present invention to provide a novel system for modifying the fluid environment of an ear.

It is another object of the present invention to provide a system as described above that addresses at least some of the shortcomings associated with existing systems for modifying the fluid environment of an ear.

It is still another object of the present invention to provide a system as described above that is compact, that has a minimal number of parts, that is relatively inexpensive to manufacture, and that is easy to wear and to operate.

Therefore, according to one aspect of the invention, there is provided a system for modifying the fluid environment of an ear, the system comprising (a) an earpiece, the earpiece being adapted to be mounted in an ear canal, the earpiece comprising a first fluid delivery path and a first fluid removal path; and (b) a gas source for supplying a gas; (c) wherein the gas source is fluidly coupled to the first fluid delivery path of the earpiece, whereby gas emitted from the first fluid delivery path causes fluid in the ear to be removed through the first fluid removal path.

In a more detailed feature of the invention, the gas source may comprise an electrochemical gas generating device, the electrochemical gas generating device may comprise an electrochemical gas generator, the electrochemical gas generator may comprise a first outlet through which gas produced by the electrochemical gas generator is emitted, and the first outlet of the electrochemical gas generator may be fluidly coupled to the first fluid delivery path of the earpiece.

In a more detailed feature of the invention, the system may further comprise an electronics housing, and the electrochemical gas generator may be disposed within the electronics housing.

In a more detailed feature of the invention, the electronics housing may be adapted to be mounted in the ear canal.

In a more detailed feature of the invention, the electronics housing may be directly mounted on the earpiece.

In a more detailed feature of the invention, the electronics housing may comprise a first fluid delivery path and a first fluid removal path, the first fluid delivery path of the electronics housing may be fluidly coupled at a first end to the first outlet of the electrochemical gas generator and may be fluidly coupled at a second end to the first fluid delivery path of the earpiece, and the first fluid removal path of the electronics housing may be fluidly coupled to the first fluid removal path of the earpiece.

In a more detailed feature of the invention, the system may further comprise a relief valve positioned within the first fluid removal path of the electronics housing.

In a more detailed feature of the invention, the electronics housing may be adapted to be worn outside the ear.

In a more detailed feature of the invention, the system may further comprise tubing for use in fluidly connecting the outlet of the electrochemical gas generator to the first fluid delivery path of the earpiece.

In a more detailed feature of the invention, the electrochemical gas generator may comprise a water electrolyzer.

In a more detailed feature of the invention, the gas emitted through the first outlet may comprise oxygen gas.

In a more detailed feature of the invention, the gas emitted through the first outlet may comprise hydrogen gas.

In a more detailed feature of the invention, the gas emitted through the first outlet may comprise a mixture of hydrogen gas and oxygen gas.

In a more detailed feature of the invention, the electrochemical gas generator may comprise an electrochemical oxygen concentrator.

In a more detailed feature of the invention, the electrochemical gas generator may comprise a proton exchange membrane, an anode on one face of the proton exchange membrane, a cathode on an opposing face of the proton exchange membrane, an anode current collector coupled to the anode opposite the proton exchange membrane, and a cathode current collector coupled to the cathode opposite the proton exchange membrane, and at least one of the anode current collector and the cathode current collector may comprise a through hole.

In a more detailed feature of the invention, each of the anode current collector and the cathode current collector may comprise a through hole, and the electrochemical gas generator may further comprise a vapor transport membrane coupled to the cathode current collector.

In a more detailed feature of the invention, the electrochemical gas generating device may further comprise a power source and control electronics operatively coupled to the electrochemical gas generator.

In a more detailed feature of the invention, the control electronics may comprise a current controller.

In a more detailed feature of the invention, the control electronics may further comprise an on/off switch.

In a more detailed feature of the invention, the control electronics may further comprise a battery monitor.

In a more detailed feature of the invention, the control electronics may further comprise at least one of a microprocessor, a sensor, and an alarm.

In a more detailed feature of the invention, the control electronics may further comprise a voltage regulator.

In a more detailed feature of the invention, the control electronics may further comprise a current selector switch.

In a more detailed feature of the invention, the control electronics may further comprise a microprocessor, a sensor, and an alarm.

In a more detailed feature of the invention, the earpiece may further comprise a tympanostomy tube suitable for insertion through a tympanic membrane of the ear.

In a more detailed feature of the invention, the system may further comprise at least one of a medicine delivery tube, a scope tube, and an instrument tube, and each of the medicine delivery tube, the scope tube and the instrument tube may be insertable into the earpiece.

In a more detailed feature of the invention, the system may further comprise a condensate drop-out port insertable into the earpiece.

In a more detailed feature of the invention, the system may further comprise a desiccant proximate to a distal end of the first fluid delivery path.

In a more detailed feature of the invention, the gas source may comprise a container holding a quantity of the gas.

In a more detailed feature of the invention, the system may further comprise a gas regulator fluidly connected between the gas source and the earpiece.

The present invention is also directed at a novel method for modifying the fluid environment of an ear.

Therefore, according to one aspect of the invention, there is provided a method for modification of a fluid environment of an ear, the method comprising the steps of (a) providing the system as described above; (b) implanting the earpiece in an ear; and (c) delivering gas from the gas source to the earpiece.

In a more detailed feature of the invention, oxygen gas may be emitted from the first fluid delivery path.

In a more detailed feature of the invention, hydrogen gas may be emitted from the first fluid delivery path.

In a more detailed feature of the invention, oxygen gas and hydrogen gas may be emitted from the first fluid delivery path.

Additional objects, as well as aspects, features and advantages, of the present invention will be set forth in part in the description which follows, and in part will be obvious from the description or may be learned by practice of the invention. In the description, reference is made to the accompanying drawings which form a part thereof and in which is shown by way of illustration various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. The drawings are not necessarily drawing to scale, and certain components may have undersized and/or oversized dimensions for purposes of explication. In the drawings wherein like reference numeral represents like parts:

FIG. 1 is a top perspective view of a first embodiment of a system for modifying the fluid environment of an ear, the system being constructed according to the present invention;

FIG. 2 is a top view of the system of FIG. 1;

FIG. 3 is a bottom view of the system of FIG. 1;

FIG. 4 is a section view of the system of FIG. 2 taken along line 4-4;

FIG. 5 is a section view of the system of FIG. 2 taken along line 5-5;

FIG. 6 is a partly exploded section view of the system of FIG. 1;

FIG. 7 is a top view of the electronics housing shown in FIG. 1;

FIG. 8 is an enlarged section view of the electrochemical gas generator shown in FIG. 4;

FIGS. 9(a) through 9(g) are schematic depictions of various alternative control electronics usable in the system of FIG. 1;

FIG. 10 is a simplified side view, shown partly in section, of the system of FIG. 1 positioned within an ear canal of a person;

FIG. 11 is a side view of a second embodiment of a system for modifying the fluid environment of an ear, the system being constructed according to the present invention;

FIG. 12 is a partly exploded perspective view of the system shown in FIG. 11;

FIG. 13 is a bottom view of the earpiece shown in FIG. 11;

FIG. 14 is a simplified side view, shown partly in section, of the system of FIG. 11 mounted on a person;

FIG. 15 is a top view of a third embodiment of a system for modifying the fluid environment of an ear, the system being constructed according to the present invention;

FIG. 16 is a section view of the system of FIG. 15 taken along line 16-16;

FIG. 17 is a section view of the system of FIG. 15 taken along line 17-17;

FIG. 18 is a simplified side view, shown partly in section, of the system of FIG. 15 positioned within an ear canal of a person;

FIG. 19 is a simplified side view, shown partly in section, of a fourth embodiment of a system for modifying the fluid environment of an ear, the system being constructed according to the present invention and being shown positioned within an ear of a person;

FIG. 20 is a top view of a fifth embodiment of a system for modifying the fluid environment of an ear, the system being constructed according to the present invention;

FIG. 21 is a section view of the system of FIG. 20 taken along line 21-21;

FIG. 22 is a section view of the system of FIG. 20 taken along line 22-22;

FIG. 23 is a partly exploded section view of the system shown in FIG. 21;

FIG. 24 is a partly exploded section view of the system shown in FIG. 22;

FIG. 25 is a section view of a sixth embodiment of a system for modifying the fluid environment of an ear, the system being constructed according to the present invention;

FIG. 26 is an exploded view of the system of FIG. 25;

FIG. 27 is a section view of a seventh embodiment of a system for modifying the fluid environment of an ear, the system being constructed according to the present invention;

FIG. 28 is a section view of an eighth embodiment of a system for modifying the fluid environment of an ear, the system being constructed according to the present invention;

FIG. 29 is a section view of a ninth embodiment of a system for modifying the fluid environment of an ear, the system being constructed according to the present invention;

FIG. 30 is a section view of an alternative embodiment of the electrochemical gas generator shown in FIG. 8;

FIG. 31 is a top view of a tenth embodiment of a system for modifying the fluid environment of an ear, the system being constructed according to the present invention;

FIG. 32 is a section view of the system of FIG. 31 taken along line 32-32;

FIG. 33 is a section view of the system of FIG. 31 taken along line 33-33; and

FIG. 34 is a partly schematic side view of an eleventh embodiment of a system for modifying the fluid environment of an ear, the system being constructed according to the present invention.

### DETAILED DESCRIPTION OF THE INVENTION

As noted above, the present invention is directed at a novel method and system for modifying the fluid environment of an ear. Such modification of the fluid environment of the ear may involve, for example, removing moisture from the ear and/or supplying the ear with one or more gases having a therapeutic effect. In contrast with existing approaches to modifying the fluid environment of the ear, which approaches are limited to removing moisture from the ear and which rely upon the use of a hair dryer to blow heated ambient air into an ear or which rely upon the use of a fan to blow room temperature ambient air into the ear, the present invention does not require the use of a hair dryer or fan to deliver heated or unheated ambient air to the ear. Instead, as will be described further below, the present invention preferably utilizes a gas source to supply one or more gases (e.g., oxygen gas; hydrogen gas; oxygen gas and hydrogen gas), and some or all of these gases may be delivered to an ear using an earpiece that is implanted in the ear. More specifically, in one embodiment, the gas source may comprise an electrochemical device or other device for generating, in situ, the one or more gases. In situ generation includes, but is not limited to, devices that use electrochemical, chemical, physical (e.g., molecular sieve) techniques or a combination of these techniques. In another embodiment, the gas source may comprise a container holding a preloaded quantity of one or more gases.

Electrochemical devices are particularly well-suited for the generation and delivery of one or more product gases at a controlled dose per unit time. In the present invention, which preferably involves the delivery of one or more electrochemically generated gases, and, in at least some embodiments, involves the delivery of electrochemically generated oxygen, such oxygen may be electrochemically generated via one of the following two types of reactions: (i) water electrolysis; and (ii) electrochemical oxygen concentration.

Water electrolysis is a common technique for generating oxygen and typically involves using an electrical current to convert water to oxygen and hydrogen. One way to perform water electrolysis is with a proton exchange membrane (PEM) electrolyzer. A PEM electrolyzer typically comprises a proton exchange membrane (PEM), an anode with catalyst on one face of the PEM, and a cathode with catalyst on the opposite face of the PEM, the combination of the PEM, the anode and the cathode often referred to as a membrane electrode assembly (MEA). The PEM, itself, typically comprises an ion-exchange polymer which, when humidified, allows the migration of protons therethrough. The PEM ion-exchange polymer also prevents reactants and products at each electrode from mixing. In use, power is consumed to split water molecules on one side of the MEA to form oxygen gas and protons. The protons migrate through the MEA to the other side, where they combine with electrons to form hydrogen gas. The oxygen production rate for a PEM electrolyzer is governed by and proportional to the electrical current provided and can be tailored for many applications. Water electrolysis may be desirable in certain cases as a production technique due to its high process efficiency, its product selectivity, and its inherent ability to control production rate by controlling the applied current.

Electrochemical oxygen concentration involves using an electrical current to concentrate oxygen present in air to pure oxygen. An electrochemical device designed for electrochemical oxygen concentration is often referred to as an electrochemical oxygen concentrator and may also comprise an MEA. In operation, an MEA-based electrochemical oxygen concentrator consumes electrical current to convert ambient oxygen to water at the cathode side of an MEA. The water product of this cathodic reaction then diffuses through the MEA to the anode, where water is oxidized into oxygen. The pure oxygen generated at the anode is then directed out of the electrochemical oxygen concentrator, where it can be used. The protons from the oxidized water at the anode cross the MEA again to the cathode to combine with oxygen from the air to form water vapor, whereupon the process repeats itself. The proton exchange membrane of the MEA also comprises an ion-exchange polymer which, when humidified, allows the migration of protons. The ion-exchange polymer also prevents reactants and products at each electrode from mixing, and other gases found in the ambient environment, such as nitrogen, from contaminating the pure oxygen product. The oxygen concentration rate is governed by and proportional to the electrical current provided and can be tailored for many applications.

In many instances, an electrochemical device capable of generating oxygen may alternatively use either water electrolysis or electrochemical oxygen concentration at a given time, depending on the reactants available and/or voltage and current settings, and such an electrochemical device may be tailored to be more appropriate for one reaction over the other.

As will be discussed further below, one aspect of the present invention is that one or more gases may be delivered to an ear. Such one or more gases may be used, for example, to modify the level of humidity within the ear, for example, by displacing or removing excess moisture from the ear. Additionally and/or alternatively, such one or more gases may provide some therapeutic benefit to the ear, aside from simply modifying the humidity level within the ear. For example, where oxygen is delivered to the ear, such oxygen may promote wound healing. As another example, where hydrogen is delivered to the ear, such hydrogen may have anti-inflammatory, antioxidant and/or antiapoptotic effects. Where, for example, oxygen and/or hydrogen is delivered to the ear, such oxygen and/or hydrogen may be generated electrochemically, for example, by the hydrolysis of water. Such water may include water that is present in the ambient environment and/or in the ear canal. Alternatively, where, for example, oxygen is delivered to the ear, such oxygen may be generated electrochemically by the concentration of oxygen from air or oxygen-enriched air. Such air or oxygen-enriched air may include air that is present in the ambient environment and/or oxygen-enriched air that is present in the ear canal.

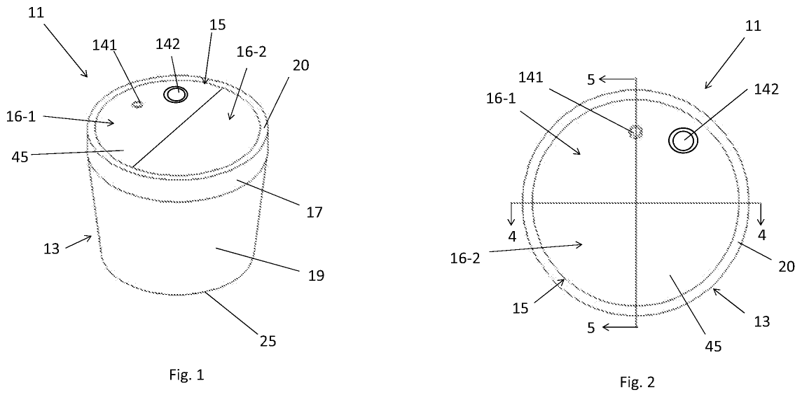

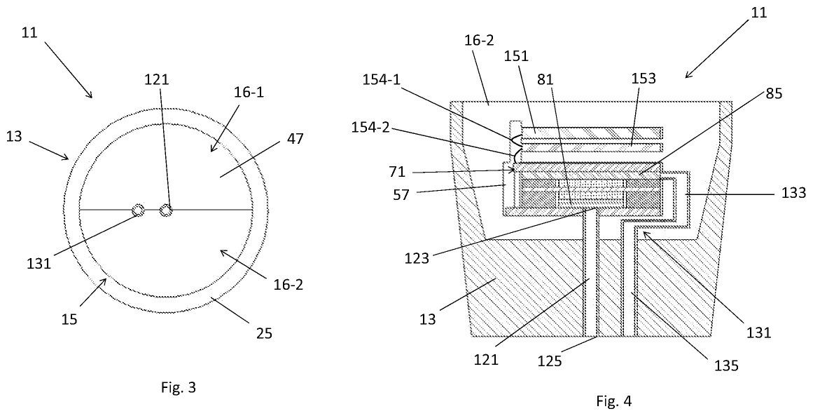

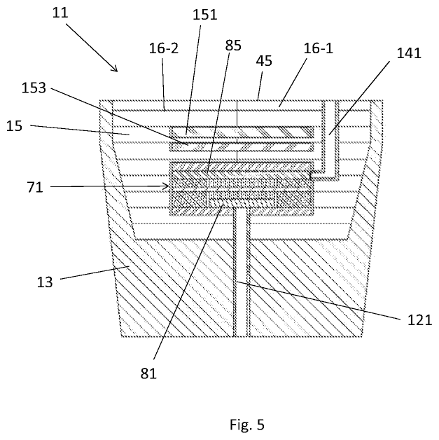

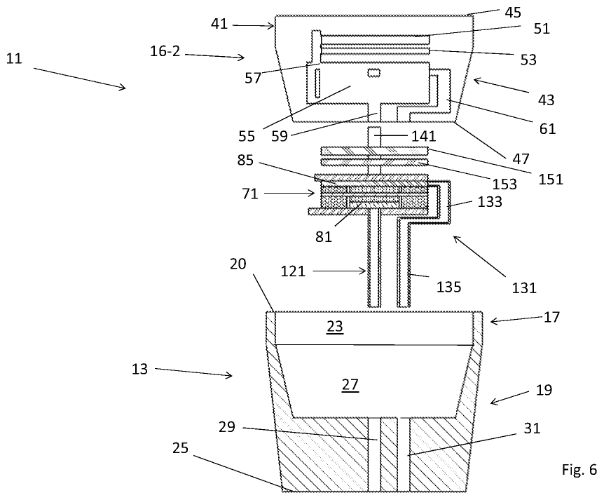

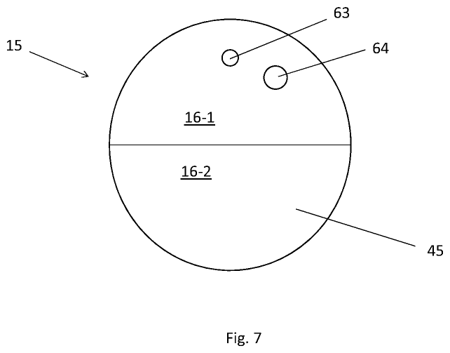

Referring now to FIGS. 1 through 6, there are shown various views of a first embodiment of a system for modifying the fluid environment of an ear, the system being constructed according to the present invention and represented generally by reference numeral 11. (For simplicity and clarity, certain components of system 11 that are not critical to the understanding of the present invention are either not shown or described herein or are shown and/or described herein in a simplified manner.)

System 11, which may be designed to be a self-contained unit removably mounted in the ear canal of a human or other subject, may comprise an earpiece 13 and an electronics housing 15.

Earpiece 13 may be a unitary (i.e., one-piece) or multi-piece structure and may be appropriately dimensioned exteriorly to be snugly, yet removably, positioned within the ear canal of a human or other subject. Earpiece 13 may have a custom shape that is fitted to a user's ear, similar to a hearing aid earmold, or may have a standard shape, similar to a hearing aid dome. Earpiece 13 may be made of or comprise one or more suitably strong, rigid, and biocompatible materials, such as, but not limited to, acrylic, silicone, polyethylene, and the like, and may be formed by machining, molding, 3D printing, and/or any other suitable manufacturing technique. In the present embodiment, earpiece 13 may comprise a proximal or top portion 17 and a distal or bottom portion 19.

Top portion 17, whose outer shape may be generally cylindrical, may terminate at a top end 20 adapted to face towards the ambient environment (i.e., in a direction away from the interior of the ear of a user). A lumen 23, which may be generally cylindrical, may be concentrically located within top portion 17 and may extend axially through the entirety of top portion 17. Bottom portion 19, whose outer shape may be generally frustoconical, may terminate at a bottom end 25 adapted to face away from the ambient environment (i.e., in a direction towards the interior of the ear of a user). A lumen 27, which may be generally frustoconical, may be concentrically disposed within bottom portion 19 and may extend axially downwardly from lumen 23 for a portion, but not the entirety, of the length of bottom portion 19.

Bottom portion 19 of earpiece 13 may further comprise a lumen 29 and a lumen 31. Lumen 29, which may be generally cylindrical and which may be considerably smaller in diameter than lumen 27, may be concentrically disposed within bottom portion 19 and may extend axially from the bottom of lumen 27 to bottom end 25. Lumen 31, which also may be generally cylindrical and considerably smaller in diameter than lumen 27, may be located off-center within bottom portion 19 and may extend axially from the bottom of lumen 27 to bottom end 25. Each of lumens 29 and 31 may be in fluid communication with lumen 27. As will be discussed further below, lumen 29 may be used in forming a first fluid delivery path to help conduct fluid in a direction towards the interior of the ear of a user, and lumen 31 may be used in forming a first fluid removal path to help conduct fluid in a direction away from the interior of the ear of a user.

Electronics housing 15, which is also shown separately in FIG. 7, may comprise two complementary half units 16-1 and 16-2, which may be permanently or reversibly joined together by one or more adhesives, fasteners, or other suitable means. Electronics housing 15 may be appropriately dimensioned exteriorly to be securely mounted within earpiece 13, with electronics housing 15 preferably mating with the space collectively defined by lumens 23 and 27 of earpiece 13. Although not shown, electronics housing 15 may be permanently or removably joined to earpiece 13 by one or more of adhesives, over-molding, mechanical fasteners (such as screws), mating threads, or any other appropriate means. Electronics housing 15 may be made of or comprise one or more suitably strong, rigid, and biocompatible materials, such as, but not limited to, acrylic, titanium, acetal resin (e.g., DELRIN® acetal homopolymer (polyoxymethylene (POM)), DuPont de Nemours, Inc., Wilmington, DE), and the like, and may be formed by machining, molding, 3D printing, and/or any other suitable manufacturing technique.

In the present embodiment, each of half units 16-1 and 16-2 may comprise a proximal or top portion 41 and a distal or bottom portion 43. Top portion 41, which has a top end 45 adapted to face towards the ambient environment (i.e., in a direction away from the interior of the ear of a user), may be generally semi-cylindrical so that, when half units 16-1 and 16-2 are assembled, the joined top portions 41 may be dimensioned to mate with lumen 23. Bottom portion 43, which has a bottom end 47 adapted to face away from the ambient environment (i.e., in a direction towards the interior of the ear of a user), may be generally semi-frustoconical so that, when half units 16-1 and 16-2 are assembled, the joined bottom portions 43 may be dimensioned to mate with lumen 27.

Electronics housing 15 may be shaped to include one or more cavities, which may be formed by the joining of half units 16-1 and 16-2. For example, in the present embodiment, electronics housing 15 may be shaped to include a power source cavity 51, a control electronics cavity 53, an electrochemical device cavity 55, a wiring cavity 57, a fluid delivery cavity 59, a first fluid removal cavity 61, a second fluid removal cavity 63, and an ambient reactant lumen 64. Fluid delivery cavity 59 may be used in forming a fluid delivery path. First fluid removal cavity 61 and second fluid removal cavity 63 may be used in forming a fluid removal path through electronics housing 15. Additional information regarding the roles of the various cavities of electronics housing 15 will be discussed further below.

It is to be understood that the respective sizes and shapes of earpiece 13 and electronics housing 15, as well as the various lumens and cavities therein, may be modified as desired.

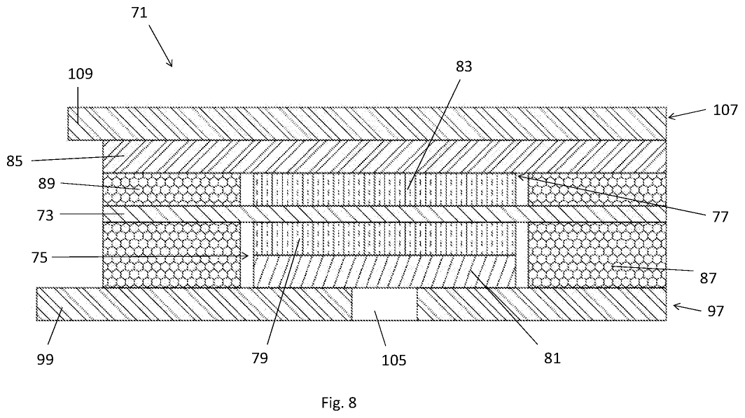

System 11 may further comprise an electrochemical gas generator 71. Electrochemical gas generator 71, which is also shown separately in FIG. 8, may be used to generate one or more product gases (e.g., oxygen gas and/or hydrogen gas). Electrochemical gas generator 71, which may be operated as a water electrolyzer or as an electrochemical oxygen generator, may be permanently or removably disposed within electrochemical device cavity 55.

Electrochemical gas generator 71 may comprise a solid polymer electrolyte membrane (PEM) 73 (also known in the art as a proton exchange membrane). PEM 73 is preferably a non-porous, ionically-conductive, electrically-non-conductive, liquid permeable and substantially gas-impermeable membrane. PEM 73 may consist of or comprise a homogeneous perfluorosulfonic acid (PFSA) polymer. Said PFSA polymer may be formed by the copolymerization of tetrafluoroethylene and perfluorovinylether sulfonic acid. See e.g., U.S. Pat. No. 3,282,875, inventors Connolly et al., issued Nov. 1, 1966; U.S. Pat. No. 4,470,889, inventors Ezzell et. al., issued Sep. 11, 1984; U.S. Pat. No. 4,478,695, inventors Ezzell et. al., issued Oct. 23, 1984; and U.S. Pat. No. 6,492,431, inventor Cisar, issued Dec. 10, 2002, all of which are incorporated herein by reference in their entireties. A commercial embodiment of a PFSA polymer electrolyte membrane is manufactured by The Chemours Company FC, LLC (Fayetteville, N.C.) as NAFION™ extrusion cast PFSA polymer membrane.

PEM 73 may be a generally planar unitary structure in the form of a continuous film or sheet. In the present embodiment, when viewed from above or below, PEM 73 may have a general circular shape. Moreover, the overall shape of electrochemical gas generator 71, when viewed from above or below, may correspond generally to the shape of PEM 73. However, it is to be understood that PEM 73, as well as electrochemical gas generator 71 as a whole, is not limited to a generally circular shape and may have a generally rectangular, annular, or other suitable shape.

Electrochemical gas generator 71 may further comprise an anode 75 and a cathode 77. Anode 75 and cathode 77 may be positioned along two opposing major faces of polymer electrolyte membrane 73. In the present embodiment, anode 75 is shown positioned along the bottom face of PEM 73, and cathode 77 is shown positioned along the top face of PEM 73; however, it is to be understood that the positions of anode 75 and cathode 77 relative to PEM 73 could be reversed.

Anode 75, in turn, may comprise an anode electrocatalyst layer 79 and an anode support 81. Anode electrocatalyst layer 79 may be positioned in direct contact with PEM 73, and, in the present embodiment, is shown as being positioned directly below and in contact with the bottom side of PEM 73. Anode electrocatalyst layer 79 defines the electrochemically active area of anode 75 and preferably is sufficiently porous and electrically- and ionically-conductive to sustain a high rate of surface oxidation reaction. Anode electrocatalyst layer 79, which may be an anode electrocatalyst layer of the type conventionally used in a PEM-based water electrolyzer, may comprise electrocatalyst particles in the form of a finely divided electrically-conductive and, optionally, ionically-conductive material (e.g., a metal powder) which can sustain a high rate of electrochemical reaction. The electrocatalyst particles may be distributed within anode electrocatalyst layer 79 along with a binder, which is preferably ionically-conductive, to provide mechanical fixation.

Anode support 81, which may be an anode support of the type conventionally used in a PEM-based water electrolyzer and may be, for example, a film or sheet of porous titanium, preferably is sufficiently porous to allow fluid (gas and/or liquid) transfer between anode electrocatalyst layer 79 and fluid delivery cavity 59 (or tubing positioned within fluid delivery cavity 59). To this end, anode support 81 may have pore sizes on the order of, for example, approximately 0.001-0.5 mm. Anode support 81 may also contain macroscopic channel features, for example, on the order of 0.2-10 mm to further assist in fluid distribution. In addition, anode support 81 is preferably electrically-conductive to provide electrical connectivity between anode electrocatalyst layer 79 and an anode current collector to be discussed below. Anode support 81 is also preferably ionically-non-conductive. Anode support 81 may be positioned in direct contact with anode electrocatalyst layer 79 and, in the present embodiment, is shown as being positioned directly below anode electrocatalyst layer 79 such that anode electrocatalyst layer 79 may be sandwiched between and in contact with PEM 73 and anode support 81. Anode support 81 may be dimensioned to entirely cover a surface (e.g., the bottom surface) of anode electrocatalyst layer 79, and, in fact, anode 75 may be fabricated by depositing anode electrocatalyst layer 79 on anode support 81.

Cathode 77 may comprise a cathode electrocatalyst layer 83 and a cathode support 85. Cathode electrocatalyst layer 83 may be positioned in direct contact with PEM 73, and, in the present embodiment, is shown as being positioned directly above and in contact with the top of PEM 73. Cathode electrocatalyst layer 83 defines the electrochemically active area of cathode 77 and preferably is sufficiently porous and electrically- and ionically-conductive to sustain a high rate of surface reduction reaction. Cathode electrocatalyst layer 83, which may be a cathode electrocatalyst layer of the type conventionally used in a PEM-based water electrolyzer, may comprise electrocatalyst particles in the form of a finely divided electrically-conductive and, optionally, ionically-conductive material (e.g., a metal powder) which can sustain a high rate of electrochemical reaction. The electrocatalyst particles may be distributed within cathode electrocatalyst layer 83 along with a binder, which is preferably ionically-conductive, to provide mechanical fixation. The reactants and products involved at anode 75 and cathode 77 may implicate ionic species that are mobile throughout the electroactive surface; therefore, an ionically-conductive medium comprising PEM 73 and, optionally, one or more ionically-conductive catalyst binders in electrocatalyst layers 79 and 83 may couple the electrodes and may allow ions to flow in support of the overall reaction electrochemistry.

Cathode support 85, which may be a cathode support of the type conventionally used in a PEM-based water electrolyzer and may be, for example, a film or sheet of porous carbon, preferably is sufficiently porous to allow fluid (gas and/or liquid) transfer between cathode electrocatalyst layer 83 and second fluid removal cavity 63 (or tubing positioned within second fluid removal cavity 63). To this end, cathode support 85 may have pore sizes on the order of, or example, approximately 0.001-0.5 mm. Cathode support 85 may also contain macroscopic channel features, for example, on the order of 0.2-10 mm to further assist in fluid distribution. In addition, cathode support 85 is electrically-conductive to provide electrical connectivity between cathode electrocatalyst layer 83 and a cathode current collector to be discussed below. Cathode support 85 is also preferably ionically-non-conductive. Cathode support 85 may be positioned in direct contact with cathode electrocatalyst layer 83 and, in the present embodiment, is shown as being positioned directly above cathode electrocatalyst layer 83 such that cathode electrocatalyst layer 83 may be sandwiched between and in contact with PEM 73 and cathode support 85. Cathode support 85 may be dimensioned to entirely cover a surface (e.g., the top surface) cathode electrocatalyst layer 83, and, in fact, cathode 77 may be fabricated by depositing cathode electrocatalyst layer 83 on cathode support 85.

The combination of PEM 73, anode 75, and cathode 77, or the combination of PEM 73, anode electrocatalyst layer 79, and cathode electrocatalyst layer 83 may be regarded collectively as a membrane-electrode assembly (MEA).

Electrochemical gas generator 71 may further comprise an anode seal 87 and a cathode seal 89. Anode seal 87, which may be an anode seal of the type conventionally used in a PEM-based water electrolyzer, may be a generally annular or frame-like member mounted around the periphery of anode 75 in a fluid-tight manner. (Anode seal 87 may be positioned in direct contact with the periphery of anode 75 or there may be a small gap between anode seal 87 and the periphery of anode 75 to facilitate assembly.) Anode seal 87, which may be made of polytetrafluoroethylene (PTFE), ethylene-propylene-diene-monomer (EPDM) rubber, or another similarly suitable material, may be ionically-non-conductive and electrically non-conductive. Anode seal 87 may also be non-porous and fluid-impermeable.

Cathode seal 89, which may be a cathode seal of the type conventionally used in a PEM-based water electrolyzer, may be a generally annular or frame-like member mounted around the periphery of cathode 77 in a fluid-tight manner. (Cathode seal 89 may be positioned in direct contact with the periphery of cathode 77 or there may be a small gap between cathode seal 89 and the periphery of cathode 77 to facilitate assembly.) Cathode seal 89, which may be made of polytetrafluoroethylene (PTFE), ethylene-propylene-diene-monomer (EPDM) rubber, or another similarly suitable material, may be ionically-non-conductive and electrically-non-conductive. Cathode seal 89 may also be non-porous and fluid-impermeable.

In the present embodiment, anode 75 and anode seal 87 may be dimensioned to jointly match the footprint of the bottom surface of PEM 73. In addition, cathode support 85, cathode catalyst layer 83, and cathode seal 89 may also be dimensioned to jointly match the footprint of the top surface of PEM 73. Notwithstanding the above, it is to be understood that the footprints of the foregoing components may be varied from what is described above.

Electrochemical gas generator 71 may further comprise an anode current collector 97. Anode current collector 97 may be similar to an anode current collector of the type conventionally used in a PEM-based water electrolyzer and may comprise, for example, a platinum-coated titanium sheet. When viewed from below, anode current collector 97 may have a footprint that substantially matches the collective footprints of anode 75 and anode seal 87, except that anode current collector 97 may additionally comprise a tab 99 that may extend radially outwardly a short distance beyond the periphery of anode seal 87 and that may be used as a terminal. Anode current collector 97 may also comprise a through hole 105, the purpose of which will become apparent below.

Electrochemical gas generator 71 may further comprise a cathode current collector 107, which may comprise a cathode current collector of the type conventionally used in a PEM-based water electrolyzer and may be, for example, a platinum-coated titanium sheet. When viewed from below, cathode current collector 107 may have a footprint that substantially matches the collective footprints of cathode 77 and cathode seal 89, except that cathode current collector 107 may additionally comprise a tab 109 that may extend radially outwardly a short distance beyond the footprint of cathode seal 89 and that may be used as a terminal.

Although not shown, electrochemical gas generator 71 may further comprise other components commonly found in conventional PEM-based water electrolyzers. For example, the static forces upon electrochemical gas generator 71 that may be required to compress anode seal 87 and cathode seal 89 to sustain good electrical contact of the serial components of electrochemical gas generator 71 and to achieve good sealing of the cell perimeter may be established and maintained using a variety of conventional fixturing or joining implements and techniques about the internal or external periphery of the assembly. Such implements may include, for instance, fasteners (e.g., screws, rivets, etc.) which may clamp endplates at either end of the serial components, or adhesives, cements, or welds which cohere the elements together in the seal region. Such implements and techniques are known to those of ordinary skill in the art. Electrochemical gas generator 71 may be operated at a range of currents, voltages and flow rates as is possible with an electrochemical oxygen generator and may be operated continuously or intermittently or via a feedback control mechanism to meet the needs of the application.

System 11 may further comprise a fluid delivery tube 121. Fluid delivery tube 121 may be a straight tube of uniform diameter that may be made of or comprise one or more rigid, biocompatible materials. Fluid delivery tube 121 may be shaped to include a top end 123 and a bottom end 125. Top end 123 of fluid delivery tube 121 may be permanently or removably mounted within through hole 105 of electrochemical gas generator 71 and may be in direct contact with, or spaced a short distance from, anode support 81. Bottom end 125 of fluid delivery tube 125 may be permanently or removably mounted within lumen 29 of earpiece 13 and may be flush with, or spaced a short distance from, bottom end 25 of earpiece 13. The portions of fluid delivery tube 125 that are intermediate to top end 123 and bottom end 125 may extend through fluid delivery cavity 59 of electronics housing 15 and lumen 29 of earpiece 13. In this manner, product gas (e.g., oxygen gas) produced at anode 75 may be conducted through electronics housing 15 and earpiece 13 using fluid delivery tube 121 and may be dispensed from system 11 in a direction towards the interior of the ear of the user. Notwithstanding the above, it is to be understood that one could omit fluid delivery tube 121 from system 11 and could simply allow product gas that is produced at anode 75 to flow along a path defined by through hole 105, fluid delivery cavity 59, and lumen 29. Also, although not shown, fluid delivery tube 121 may be equipped with filters and/or check valves to prevent electrochemical gas generator 71 from becoming contaminated by biological materials or from condensate flow backwards into the electrochemical gas generator 71.

System 11 may further comprise a first fluid removal tube 131. First fluid removal tube 131 may be a tube of uniform diameter that may be made of or comprise one or more rigid, biocompatible materials. First fluid removal tube 131 may be shaped to include a proximal portion 133 and a distal portion 135. Proximal portion 133 may be appropriately sized and shaped to be permanently or removably mounted within first fluid removal cavity 61 of electronics housing 15 and may have a proximal end in direct contact with, or spaced a short distance from, cathode support 85. Distal portion 135 may be appropriately sized and shaped to be permanently or removably mounted within lumen 31 of earpiece 13 and may have a distal end flush with, or spaced a short distance from, bottom end 25 of earpiece 13. In this manner, moisture and other fluids present within an ear may be drawn into system 11 (aided by the current produced by the flow of oxygen released from system 11) and may be conducted through first fluid removal tube 131 to cathode support 85. Notwithstanding the above, it is to be understood that one could omit first fluid removal tube 131 from system 11 and could simply allow fluids present within the ear to flow along a path defined by lumen 31 and first fluid removal cavity 61. Also, although not shown, first fluid removal tube 131 may be equipped with filters or check valves to prevent electrochemical gas generator 71 from becoming contaminated by biological materials or other materials drawn into the electrochemical gas generator 71. It may be noted that, where electrochemical gas generator 71 is used as an electrochemical oxygen concentrator using oxygen present in the ear canal distal to system 11, first fluid removal tube 131 may also serve to deliver reactant oxygen to cathode support 85.

System 11 may further comprise a second fluid removal tube 141. Second fluid removal tube 141 may be a tube of uniform diameter that may be made of or comprise one or more rigid, biocompatible materials. Second fluid removal tube 141 may be appropriately sized and shaped to be permanently or removably mounted within second fluid removal cavity 63 of electronics housing 15 and may have a distal end in direct contact with, or spaced a short distance from, cathode support 85 and a proximal end flush with, or spaced a short distance from, top end 45 of electronics housing 15. In this manner, moisture and other fluids present within cathode support 85 (particularly fluids conducted from the ear to cathode support 85 using first fluid removal tube 131) may be conducted proximally through second fluid removal tube 141 and released to the ambient environment outside of the ear. Notwithstanding the above, it is to be understood that one could omit second fluid removal tube 141 from system 11 and could simply allow fluids from cathode support 85 to flow proximally along a path defined by second fluid removal cavity 63.

System 11 may further comprise an ambient reactant delivery tube 142. Ambient reactant delivery tube 142 may be a tube of uniform diameter that may be made of or comprise one or more rigid, biocompatible materials. Ambient reactant delivery tube 142 may be appropriately sized and shaped to be permanently or removably mounted within ambient reactant lumen 64 of electronics housing 15 and may have a distal end in direct contact with, or spaced a short distance from, cathode support 85 and a proximal end flush with, or spaced a short distance from, top end 45 of electronics housing 15. In this manner, where, for example, electrochemical gas generator 71 is operated as a water electrolyzer using water or water vapor present in ambient air, such ambient air may pass distally through ambient reactant delivery tube 142 to cathode support 85. Water or water vapor present in the ambient air may then pass through cathode electrocatalyst layer 83 and PEM 73 to anode electrocatalyst layer 79, where it may undergo hydrolysis to form oxygen. Alternatively, where, for example, electrochemical gas generator 71 is operated as an electrochemical oxygen concentrator using oxygen present in ambient air, such ambient air may pass distally through ambient reactant delivery tube 142 to cathode support 85. Oxygen present in the ambient air may then pass to cathode electrocatalyst layer 83, where it may be converted to water as part of the typical electrochemical oxygen concentration process. Where, for example, electrochemical gas generator 71 is operated as an electrochemical oxygen concentrator, but it is desired to exclusively use air from the ear that is distal to system 11 as a source of reactant oxygen, one may plug the proximal end of ambient reactant delivery tube 142 to prevent generated oxygen from escaping therethrough.

It is to be understood that one could omit ambient reactant delivery tube 142 from system 11 and could simply allow ambient fluids to flow distally to cathode support 85 along a path defined by ambient reactant lumen 64. Also, it is to be understood that, if electrochemical gas generator 71 is to be used exclusively as a water electrolyzer, one could modify ambient reactant lumen 64 and ambient reactant delivery tube 142 so that they lead to anode support 81, as opposed to cathode support 85 (provided that PEM 73 can be kept sufficiently humidified).

System 11 may further comprise a power source 151, which may be permanently or removably mounted within power source cavity 51 of electronics housing 15. Power source 151, which may be used to power electrochemical gas generator 71, may be a primary or rechargeable battery or may be any other type of similarly suitable power source. Although power source 151 is shown as having a generally cylindrical shape, it is to be understood that the shape of power source 151 can be cuboid or any other suitable shape. Acceptable battery chemistries and battery packagings may be any that are safe for use near the ear or within the ear canal. Acceptable batteries may include, but are not limited to, zinc-air primary batteries of the type that are commonly used in hearing aids. Power source 151 may be replaced or recharged during patient use. Power source 151 may include energy harvesting (also called ambient energy) technologies from the wearable electronics field.

System 11 may further comprise control electronics 153, which may be permanently or removably mounted within control electronics cavity 53 of electronics housing 15 and which may be used to control the operation of electrochemical gas generator 71. As seen best in FIG. 4, power source 151 may be electrically connected by one or more wires 154-1 to control electronics 153. Control electronics 153, in turn, may be electrically connected to electrochemical gas generator 71 via one or more wires 154-2. Wires 154-1 and 154-2 may be positioned within wiring cavity 57 of electronics housing 15.

Control electronics 153 may comprise a wide array of control features. For example, and without limitation, in one embodiment, control electronics 153 may comprise an on/off switch that may be used to control when electrochemical gas generator 71 operates or may include a simple circuit that begins operation when the power source is installed and ends operation when the power source runs out of power and/or is removed from the device. In another embodiment, control electronics 153 may include a circuit that provides a constant current to electrochemical gas generator 71 or may include a circuit that provides a constant voltage that is converted to a current and provided to electrochemical gas generator 71. In another embodiment, control electronics 153 may include circuitry that decreases applied current to electrochemical gas generator 71 and, hence, oxygen production when the power source reaches a low level in order to extend oxygen production life. In another embodiment, control electronics 153 may incorporate power monitoring circuitry and a low battery alarm that may provide an audible, visual or motion signal to the user, caregiver or physician.

In another embodiment, control electronics 153 may interface with one or more sensors including, but not limited to, pressure sensors, humidity sensors, voltage sensors, gas sensors, flow sensors, and accelerometers. Control electronics 153 may use such sensors to provide feedback control to control some aspect of the operation of electrochemical gas generator 71. These aspects may include on/off or current level. In another embodiment, control electronics 153 may have a switch that allows a physician to set one of several preprogrammed flow rates to adjust the device based on a desired flow rate dependent upon a patient's ear condition or body size. Control electronics 153 may include an electronic mechanism to provide the current set points for such flow rates.

In another embodiment, control electronics 153 may include a microprocessor or may include analog electronics without the use of a microprocessor. In another embodiment, control electronics 153 may provide a higher start-up current for a period of time to flush a tubing system and/or the ear canal, or control electronics 153 may provide for intermittent provision of oxygen to meet a therapeutic need or to conserve energy. In another embodiment, control electronics 153 may include a relative humidity sensor that detects when an optimal humidity has been reached, and a relative humidity sensor reading may activate an alarm to indicate that optimal humidity has been reached and that the device can be removed from the ear or a relative humidity sensor reading may shut down the device when an optimal humidity has been reached and may restart the device when the relative humidity is outside the optimal range.

In another embodiment, control electronics 153 may include an electrochemical cell voltage sensor, and the voltage sensor, when operating in an electrolyzer, may detect when the voltage is rising, indicative of nearly dry conditions in the ear. In another embodiment, a voltage sensor reading may activate an alarm to indicate that optimal humidity has been reached and that the device can be removed from the ear, or a voltage sensor reading may shut down the device when an optimal humidity has been reached and may restart the device when the relative humidity is outside the optimal range. In another embodiment, the current of the electrochemical gas generator 71 and the resulting oxygen flow rate may be specifically adjusted to modify the fluid environment of the ear to a certain oxygen or humidity level.

It is to be understood that the various illustrative embodiments of control electronics 153 set forth above are not mutually exclusive and may be combined in various ways.

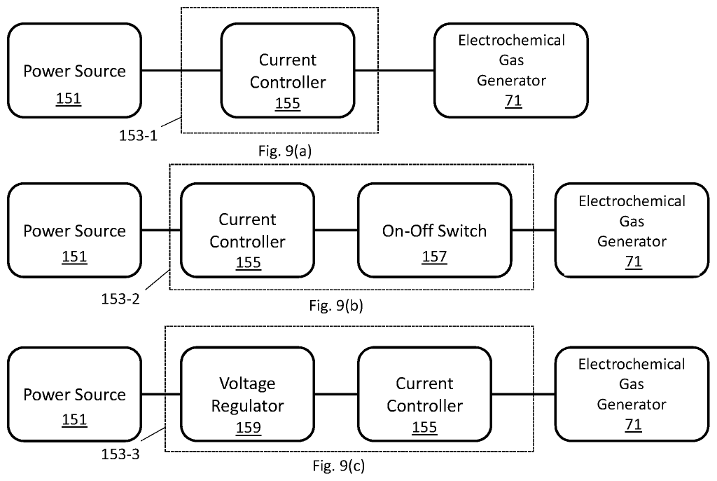

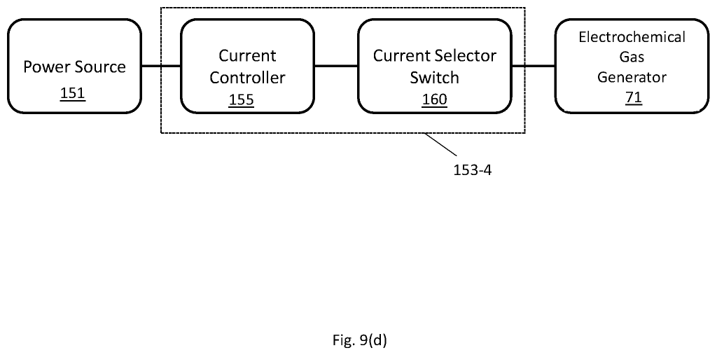

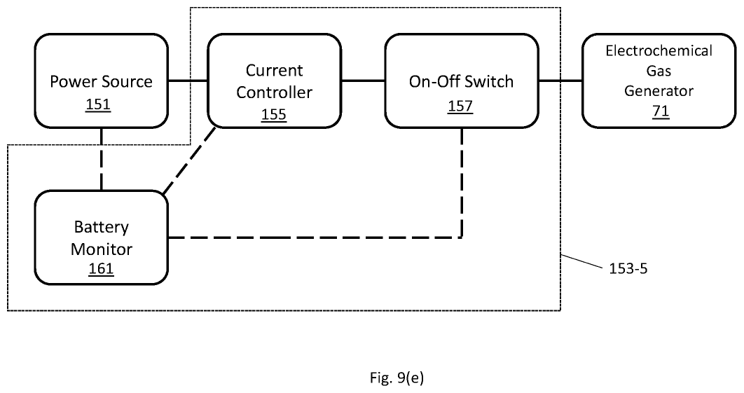

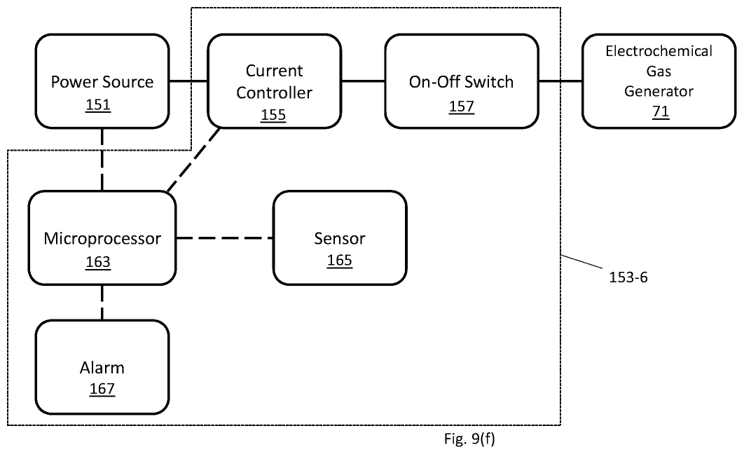

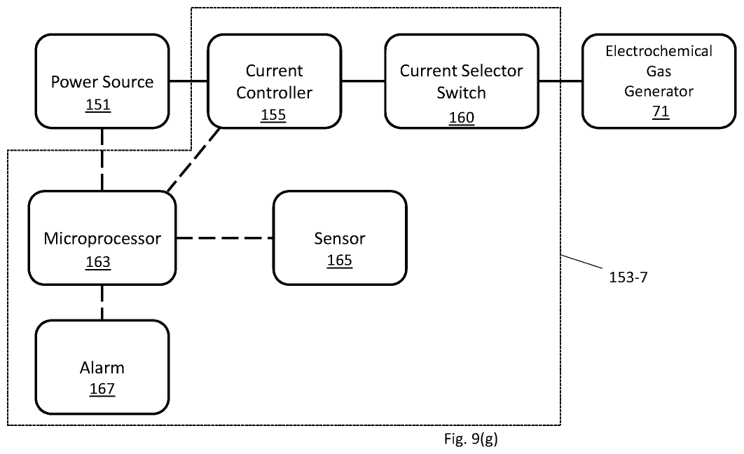

For example, FIGS. 9(a) through 9(g) schematically depict a number of alternative examples of control electronics 153, such examples being depicted using reference numerals 153-1 through 153-7, respectively. More specifically, FIG. 9(a) shows control electronics 153-1 in the form of a current controller 155, wherein current controller 155 is coupled between power source 151 and electrochemical gas generator 71. FIG. 9(b) shows control electronics 153-2 in the form of current controller 155 and an on-off switch 157, wherein current controller 155 is coupled between power source 151 and on-off switch 157, and wherein on-off switch 157 is coupled between current controller 155 and electrochemical gas generator 71. FIG. 9(c) shows control electronics 153-3 in the form of a voltage regulator 159 and current controller 155, wherein voltage regulator 159 is coupled between power source 151 and current controller 155, and wherein current controller 155 is coupled between voltage regulator 159 and electrochemical gas generator 71. FIG. 9(d) shows control electronics 153-4 in the form of current controller 155 and current selector switch 160, wherein current controller 155 is coupled between power source 151 and current selector switch 160, and wherein current selector switch 160 is coupled between current controller 155 and electrochemical gas generator 71.

FIG. 9(e) shows control electronics 153-5 in the form of current controller 155, on-off switch 157, and battery monitor 161. Current controller 155 is coupled between power source 151 and on-off switch 157, and on-off switch 157 is coupled between current controller 155 and electrochemical gas generator 71. Battery monitor 161 is coupled to each of power source 151, current controller 155, and on-off switch 157 and regulates the operation of current controller 155 and on-off switch 157 based on monitored readings of power source 151. FIG. 9(f) shows control electronics 153-6 in the form of current controller 155, on-off switch 157, microprocessor 163, sensor 165, and alarm 167. Current controller 155 is coupled between power source 151 and on-off switch 157, and on-off switch 157 is coupled between current controller 155 and electrochemical gas generator 71. A microprocessor 163 is coupled to each of current controller 155, a sensor 165, and an alarm 167 and controls the operation of each based on readings from power source 151. FIG. 9(g) shows control electronics 153-7 in the form of current controller 155, current selector switch 160, microprocessor 163, sensor 165, and alarm 167. Current controller 155 is coupled between power source 151 and current selector switch 160, and current selector switch 160 is coupled between current controller 155 and electrochemical gas generator 71. A microprocessor 163 is coupled to each of current controller 155, a sensor 165, and an alarm 167 and controls the operation of each based on readings from power source 151.

The combination of electrochemical gas generator 71 and power source 151 or the combination of electrochemical gas generator 71, power source 151, and control electronics 153 may be regarded as an electrochemical gas generating device.

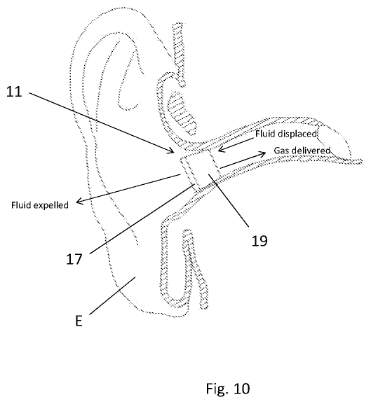

Referring now to FIG. 10, system 11 is shown implanted in an ear E of a person. Although ear E is shown as a right ear, it is to be understood that system 11, as well as all other systems disclosed herein, may be used in either a right ear or a left ear. As can be seen, when in use, system 11 may generate one or more product gases (e.g., oxygen gas, hydrogen gas), which may then be released at the distal end (i.e., eardrum-facing end) of system 11. The one or more product gases released from system 11 may cause moisture and/or other fluid that is present within the portion of the ear canal that is distal to system 11 to be swept into system 11 and, thereafter, to be discharged from system 11 at its proximal end, whereupon such moisture and/or other fluid may be released into the ambient environment. Accordingly, in this manner, system 11 may be used to reduce the amount of moisture and/or change the composition of fluid that is present within ear E.

Additionally, because oxygen promotes wound healing and hydrogen has anti-inflammatory, antioxidant and/or antiapoptotic effects, system 11 may be concurrently or alternatively used for the purpose of delivering oxygen and/or hydrogen to the ear canal. Consequently, although system 11 is specifically configured to release oxygen gas, system 11 could be configured to release hydrogen gas, instead of oxygen gas, or, alternatively, could be configured to release both hydrogen gas and oxygen gas.

Moreover, it is to be understood that, although system 11 is constructed so that both electronics housing 15, which houses electrochemical gas generator 71, and earpiece 13 are entirely positioned within the ear of a user, system 11 could be modified so that electronics housing 15 and/or one or more of the components disposed therewithin (e.g., electrochemical gas generator 71) may be spaced apart from earpiece 13, for example, by being positioned outside the ear of a user.

System 11 may be worn in an ear for an extended period of time and may be operated continuously while being worn (e.g., 24/7 operation). Alternatively, system 11 may be worn continuously for an extended period of time but only operated on a periodic, intermittent, or as-needed basis, or system 11 may be worn and operated when needed and removed when not needed.

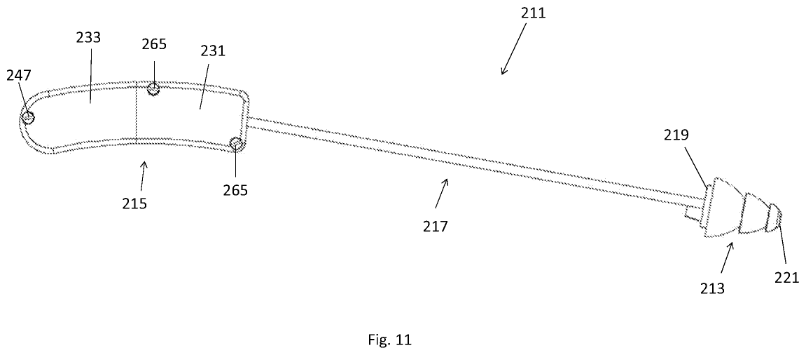

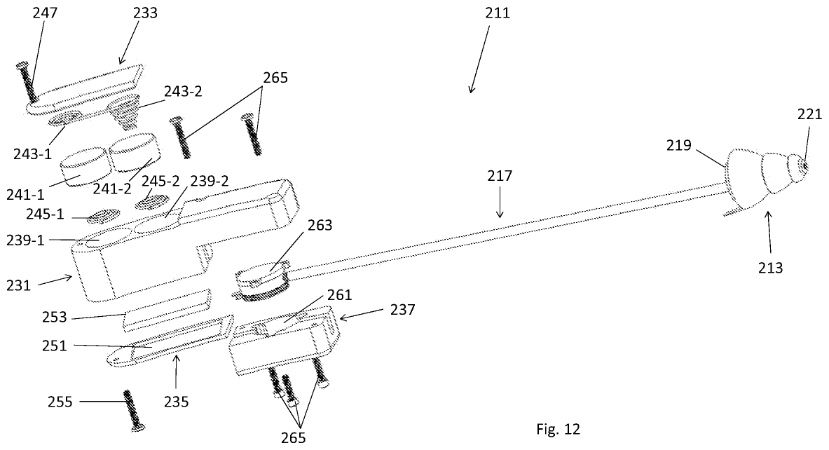

For example, referring now to FIGS. 11 and 12, there are shown various views of a second embodiment of a system for modifying the fluid environment of an ear, the system being constructed according to the present invention and represented generally by reference numeral 211. (For simplicity and clarity, certain components of system 211 that are not critical to the understanding of the present invention are either not shown or described herein or are shown and/or described herein in a simplified manner.)

System 211, which may be designed to be a self-contained unit, may comprise an earpiece 213, an electronics housing 215, and a length of tubing 217.

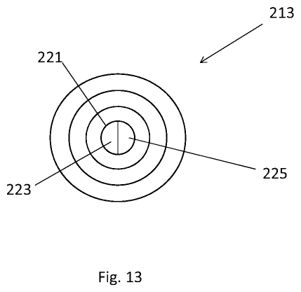

Earpiece 213, which is also shown separately in FIG. 13, may be a unitary (i.e., one-piece) or multi-piece structure and may be appropriately dimensioned exteriorly to be snugly, yet removably, positioned within the ear canal of a human or other subject. Earpiece 213 may have a custom shape that is fitted to a user's ear, similar to a hearing aid earmold, or may have a standard shape, similar to a hearing aid dome. Earpiece 213 may be made of or comprise one or more suitably strong, rigid, and biocompatible materials, such as, but not limited to, acrylic, silicone, polyethylene, and the like, and may be formed by machining, molding, 3D printing, and/or any other suitable manufacturing technique. In the present embodiment, earpiece 213 may comprise a proximal or top end 219 adapted to face towards the ambient environment (i.e., in a direction away from the interior of the ear of a user) and a distal or bottom end 221 adapted to face towards the interior of the ear of a user (i.e., away from the ambient environment).

A pair of lumens 223 and 225 may be provided in earpiece 213 and may extend axially from top end 219 to bottom end 221. As will be discussed further below, lumen 223 may be used as a fluid delivery conduit to transport fluid (e.g., oxygen gas) to the ear of a user, and lumen 225 may be used as a fluid removal conduit to transport fluid (e.g., moisture) from the ear of a user.

Electronics housing 215, which may be appropriately dimensioned to be worn on the exterior of an ear (e.g., over or behind the ear) and which may be made of the same one or more types of materials used to form electronics housing 15, may be collectively formed by a battery storage member 231, a top cover 233, a bottom cover 235, and an electrochemical gas generator storage member 237. Battery storage member 231 may be shaped to include cavities 239-1 and 239-2. Cavity 239-1 may be dimensioned to removably receive a battery 241-1, a top contact 243-1, and a bottom contact 245-1, and cavity 239-2 may be dimensioned to removably receive a battery 241-2, a top contact 243-2, and a bottom contact 245-2. Batteries 241-1 and 241-2 may be similar or identical to power source 151 of system 11.

Top cover 233, which may be used to cover the tops of cavities 239-1 and 239-2, may be secured to a top end of battery storage member 231 using a screw 247. Bottom cover 235, which may include a recess 251 for receiving a printed circuit board 253 with control electronics, may be secured to the bottom of battery storage member 231 using a screw 255.

Electrochemical gas generator storage member 237 may be shaped to include a cavity 261. Cavity 261, in turn, may be used to receive an electrochemical gas generator 263, which may be similar or identical to electrochemical gas generator 71 of system 11. Electrochemical gas generator storage member 237 may be secured to battery storage member 231 using screws 265. Also, although not shown, one or more ambient reactant delivery tubes or lumens may be appropriately provided to permit ambient air to gain access to the operative components of electrochemical gas generator 263.

Tubing 217 may be an elongated flexible structure made of or comprising one or more suitable chemically inert, biocompatible materials. A proximal end of tubing 217 may be permanently or removably coupled to an output of electrochemical gas generator 263, and a distal end of tubing 217 may be permanently or removably coupled to lumen 223 of earpiece 213. In this manner, a product gas from electrochemical gas generator 263 may be delivered through tubing 217 and earpiece 213 to the user.

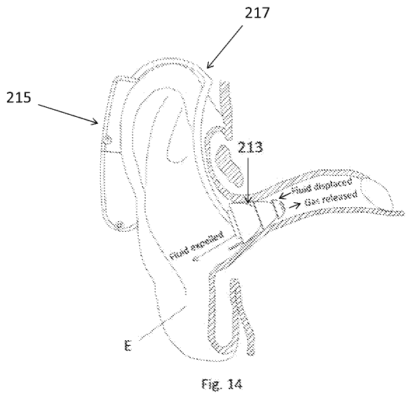

Referring now to FIG. 14, system 211 is shown in use, with earpiece 213 mounted within the ear E of a person, with electronics housing 215 mounted behind the ear E, and with tubing 217 connecting electronics housing 215 and earpiece 213. Although ear E is shown as a right ear, it is to be understood that system 211 may be used with either a right ear or a left ear. As can be seen, when in use, system 211 may generate one or more product gases (e.g., oxygen gas), which may then be released at the distal end (i.e., eardrum-facing end) of earpiece 213. The one or more product gases released from earpiece 213 may cause moisture and/or other fluid that is present in the portion of the ear canal that is distal to earpiece 213 to be swept into earpiece 213 (namely, into lumen 225) and, thereafter, to be discharged from earpiece 213 at its proximal end, whereupon such moisture and/or other fluid is released into the ambient environment. Accordingly, in this manner, system 211 may be used to reduce the amount of moisture and/or change the composition of fluid that is present within ear E.

Additionally, because oxygen promotes wound healing and hydrogen has anti-inflammatory, antioxidant and/or antiapoptotic effects, system 211 may be concurrently or alternatively used for the purpose of delivering oxygen and/or hydrogen to the ear canal. Consequently, although system 211 is specifically configured to release oxygen gas, system 211 could be configured to release hydrogen gas, instead of oxygen gas, or, alternatively, could be configured to release both hydrogen gas and oxygen gas.

Moreover, it is to be understood that, although system 211 is configured for electronics housing 215 to be worn on the outside/behind an ear, electronics housing 215 may be located at any convenient location, such as on a pendant around the neck of a user or held in a hat or headband.

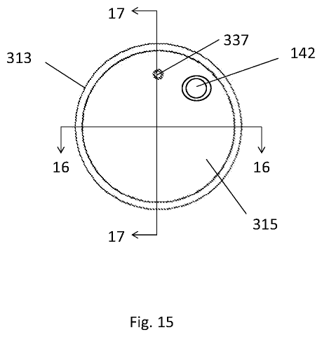

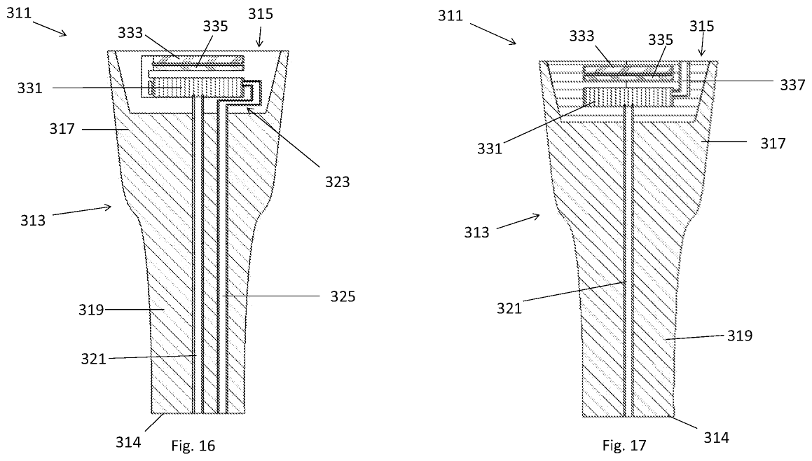

Referring now to FIGS. 15 through 17, there are shown various views of a third embodiment of a system for modifying the fluid environment of an ear, the system being constructed according to the present invention and represented generally by reference numeral 311. (For simplicity and clarity, certain components of system 311 that are not critical to the understanding of the present invention are either not shown or described herein or are shown and/or described herein in a simplified manner.)

System 311, which may be designed to be a self-contained unit removably mounted in the ear canal of a human or other subject, may comprise an earpiece 313 and an electronics housing 315.

Earpiece 313 may be similar in many regards to earpiece 13 of system 11, the primary differences between the two earpieces being their respective shapes and sizes. More specifically, earpiece 313 may be longer than earpiece 13 so that, when earpiece 313 is implanted in an ear canal, a distal end 314 of earpiece 313 may be positioned closer to the tympanic membrane of the ear. In addition, earpiece 313 may be shaped to comprise a proximal or top portion 317 and a distal or bottom portion 319. Top portion 317 may be generally frustoconical, and bottom portion 319 may taper slightly inwardly distally.

Electronics housing 315 may be similar in many regards to electronics housing 15 of system 11, the primary difference between the two housings being that electronics housing 315 may be frustoconical in shape without also including a generally cylindrical portion at its proximal end.

System 311 may comprise a fluid delivery tube 321, instead of fluid delivery tube 121, and a first fluid removal tube 323, instead of first fluid removal tube 131. Fluid delivery tube 321 may be generally similar to fluid delivery tube 121 of system 11, except that fluid delivery tube 321 may be longer than fluid delivery tube 121, this being in large part due to the greater length of earpiece 313 than earpiece 13. In a similar vein, first fluid removal tube 323 may be generally similar to first fluid removal tube 131, except that first fluid removal tube 323 may comprise a distal portion 325 that may be longer than distal portion 135 of first fluid removal tube 131, again this being in large part due to the greater length of earpiece 313 than earpiece 13.

System 311 may further comprise an electrochemical gas generator 331, which may be similar or identical to electrochemical gas generator 71 of system 11, a power source 333, which may be similar or identical to power source 151 of system 11, control electronics 335, which may be similar or identical to control electronics 153 of system 11, and a second fluid removal tube 337, which may be similar or identical to second fluid removal tube 141.

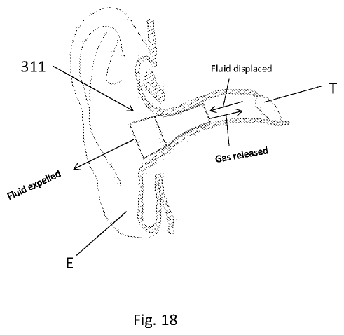

Referring now to FIG. 18, system 311 is shown implanted in an ear E of a person. Although ear E is shown as a right ear, it is to be understood that system 311 may be used in either a right ear or a left ear. As can be seen, because of its increased length, system 311 releases its product gas (e.g., oxygen gas) deeper within the ear, i.e., closer to the tympanic membrane T, than is the case with systems 11 or 211. This may be desirable in removing moisture or other fluid that is located deeper within the ear and/or in directing one or more therapeutic gases to a location deeper within the ear.

Additionally, because oxygen promotes wound healing and hydrogen has anti-inflammatory, antioxidant and/or antiapoptotic effects, system 311 may be concurrently or alternatively used for the purpose of delivering oxygen and/or hydrogen to the ear canal. Consequently, although system 311 is specifically configured to release oxygen gas, system 311 could be configured to release hydrogen gas, instead of oxygen gas, or, alternatively, could be configured to release both hydrogen gas and oxygen gas.

Moreover, it is to be understood that, although system 311 is constructed so that both electronics housing 315, which houses electrochemical gas generator 331, and earpiece 313 are entirely positioned within the ear of a user, system 311 could be modified so that electronics housing 315 and/or one or more of the components disposed therewithin (e.g., electrochemical gas generator 331) may be spaced apart from earpiece 313, for example, as in system 211.

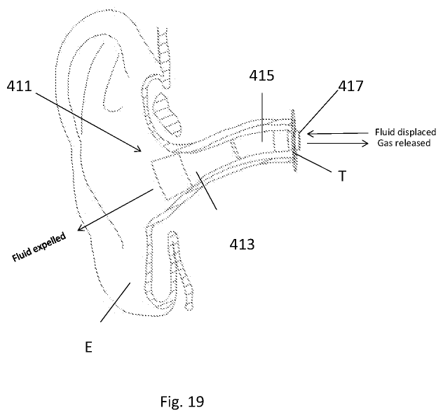

Referring now to FIG. 19, there is shown a side view of a fourth embodiment of a system for modifying the fluid environment of an ear, the system being constructed according to the present invention and represented generally by reference numeral 411, system 411 being shown implanted in an ear E of a person. (For simplicity and clarity, certain components of system 411 that are not critical to the understanding of the present invention are either not shown or described herein or are shown and/or described herein in a simplified manner.)

System 411, which may be designed to be a self-contained unit removably mounted in the ear of a human or other subject, may be similar in certain respects to system 311. For example, system 411 may comprise a proximal portion 413 that is similar in most respects to system 311. System 411 may differ from system 311 in that system 411 may further comprise an intermediate portion 415, which may extend distally from proximal portion 413, and a distal portion 417, which may extend distally from intermediate portion 415. Intermediate portion 415, which may be in the form of an extensible telescopic structure, may include fluid delivery and removal lumens that are in fluid communication with the corresponding fluid delivery and removal structures of proximal portion 413. Distal portion 417, which may be in the form of tympanostomy tube insertable through the tympanic membrane T, may include fluid delivery and removal tubes in fluid communication with those of intermediate portion 415. In this manner, gas produced by an electrochemical gas generator within proximal portion 413 may be delivered to the middle ear, and moisture and/or other fluid within the middle ear may be released outside the ear to the ambient environment.

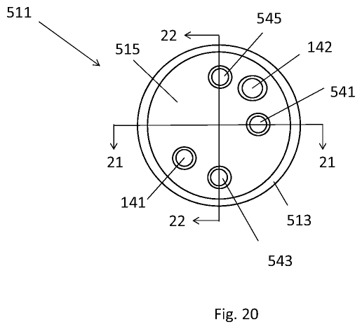

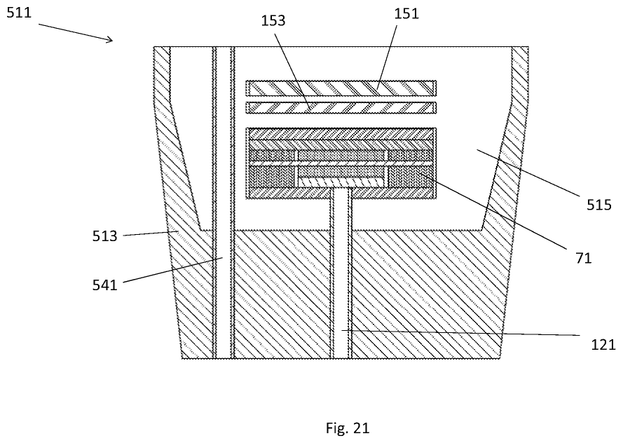

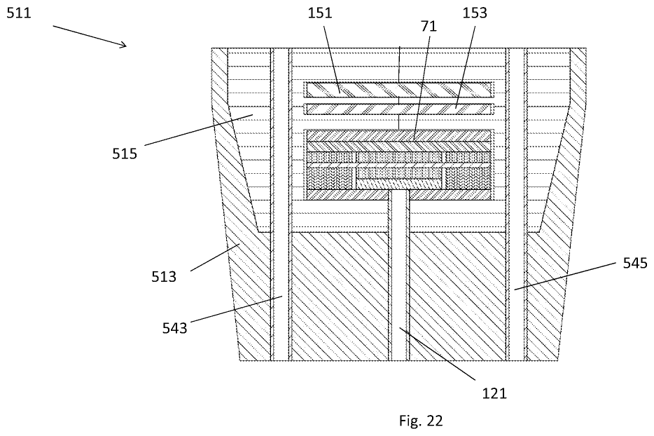

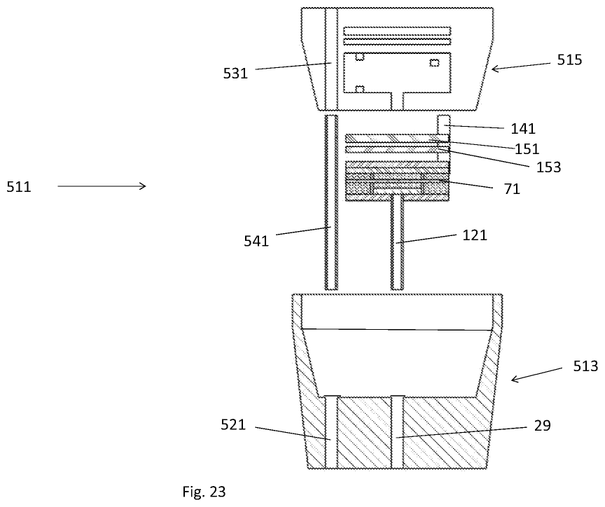

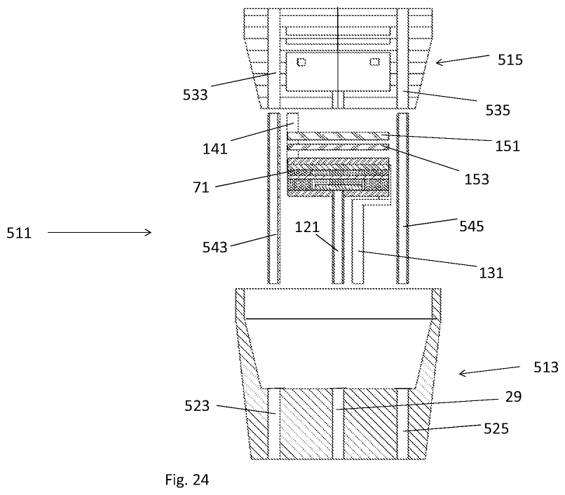

Referring now to FIGS. 20 through 24, there are shown various views of a fifth embodiment of a system for modifying the fluid environment of an ear, the system being constructed according to the present invention and represented generally by reference numeral 511. (For simplicity and clarity, certain components of system 511 that are not critical to the understanding of the present invention are either not shown or described herein or are shown and/or described herein in a simplified manner.)

System 511, which may be designed to be a self-contained unit removably mounted in the ear of a human or other subject, may be similar in most respects to system 11, the primary difference between the two systems being that system 511 may additionally comprise three optional open longitudinal ports, namely, a medicine delivery port to allow for gravity-aided administration of topical medication to the ear, a scope port to allow for observation of the ear, and an instrument port to allow for intervention in the ear, all without requiring system 511 to be removed from the ear.

More specifically, system 511 may comprise an earpiece 513 and an electronics housing 515. Earpiece 513 may be similar in most respects to earpiece 13 of system 11, the primary differences between the two earpieces being that earpiece 513 may additionally comprise a medicine delivery lumen 521, a scope lumen 523, and an instrument lumen 525 and may omit a lumen corresponding to lumen 31 of earpiece 13. Electronics housing 515 may be similar in most respects to electronics housing 15 of system 11, the primary difference between the two electronics housings being that electronics housing 515 may additionally comprise a medicine delivery lumen 531 alignable with medicine delivery lumen 521 of earpiece 513, a scope lumen 533 alignable with scope lumen 523 of earpiece 513, and an instrument lumen 535 alignable with instrument lumen 525 of earpiece 513.

System 511 may further comprise a medicine delivery tube 541, a scope tube 543, and an instrument tube 545, each of which may be made of or comprise one or more suitable biocompatible materials. Medicine delivery tube 541 may be positioned so that a proximal portion thereof may be permanently or removably mounted within medicine delivery lumen 531 of electronics housing 515 and so that a distal portion thereof may be permanently or removably mounted within medicine delivery lumen 521 of earpiece 513. Scope tube 543 may be positioned so that a proximal portion thereof may be permanently or removably mounted within scope lumen 533 of electronics housing 515 and so that a distal portion thereof may be permanently or removably mounted within scope lumen 523 of earpiece 513. Instrument tube 545 may be positioned so that a proximal portion thereof may be permanently or removably mounted within instrument lumen 535 of electronics housing 515 and so that a distal portion thereof may be permanently or removably mounted within instrument lumen 535 of earpiece 513.

It is to be understood that system 511 may be modified by removing one or more of the above-described ports and/or by removing some or all of the tubing positioned therein.

Also, it is to be understood that one or more of the ports of system 511 may be incorporated into any of the earpieces disclosed herein, regardless of whether the electronics housing is configured for placement in the ear or outside the ear.

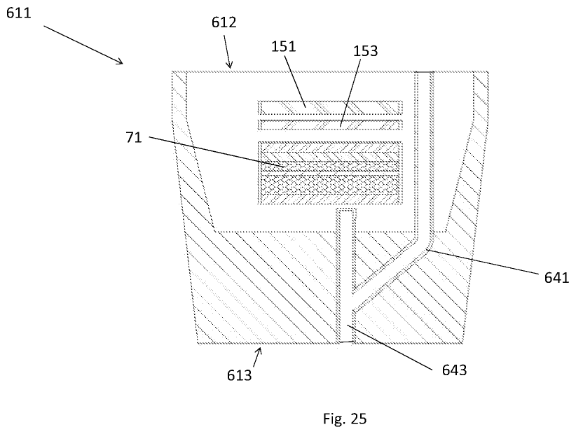

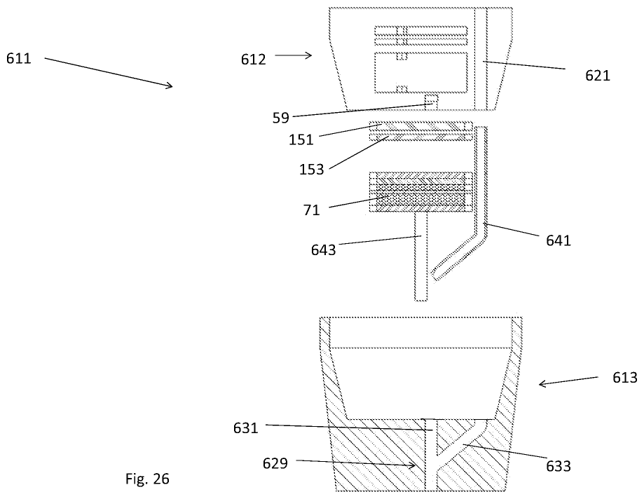

Referring now to FIGS. 25 and 26, there are shown various views of a sixth embodiment of a system for modifying the fluid environment of an ear, the system being constructed according to the present invention and represented generally by reference numeral 611. (For simplicity and clarity, certain components of system 611 that are not critical to the understanding of the present invention are either not shown or described herein or are shown and/or described herein in a simplified manner.)

System 611, which may be designed to be a self-contained unit removably mounted in the ear of a human or other subject, may be similar in most respects to system 11, the primary difference between the two systems being that system 611 may additionally comprise a condensate drop-out port to allow for gravity-aided draining of any condensate buildup without requiring the removal of system 611.

More specifically, system 611 may comprise an electronics housing 612 and an earpiece 613. Electronics housing 612 may be similar in most respects to electronics housing 15 of system 11, the primary difference between the two electronics housings being that electronics housing 612 may additionally comprise a condensate drop-out lumen 621 extending axially from its proximal or top end to its distal or bottom end.

Earpiece 613 may be similar in most respects to earpiece 13 of system 11, the primary difference between the two earpieces being that earpiece 613 may comprise a lumen 629, instead of lumen 29, wherein lumen 629 may include a first branch 631 having a proximal end alignable with cavity 59 of electronics housing 612 and a second branch 633 having a proximal end alignable with lumen 621 of electronics housing 612.

System 611 may further comprise a condensate tube 641 and a fluid delivery tube 643. Condensate tube 641, which may be made of or comprise one or more suitable biocompatible materials, may be positioned so that a proximal portion thereof may be permanently or removably mounted within lumen 621 of electronics housing 612 and so that a distal portion thereof may be permanently or removably mounted within branch 633 of earpiece 613. Fluid delivery tube 643, which may be made of or comprise one or more suitable biocompatible materials, may be positioned so that a proximal portion thereof may be permanently or removably mounted within cavity 59 of electronics housing 612 and so that a distal portion thereof may be permanently or removably mounted within branch 631 of earpiece 613. Although not shown, fluid delivery tube 643 may have a side opening to permit fluid communication with condensate tube 641.

It is to be understood that system 611 may additionally include one or more of the ports (and associated tubing) of system 511.

Also, it is to be understood that the condensate drop-out port of system 611 may be incorporated into earpieces like that of system 211, in which the electronics housing is placed outside of the ear.

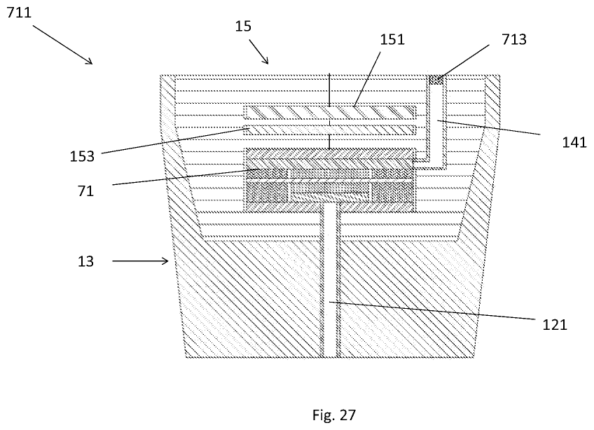

Referring now to FIG. 27, there is shown a section view of a seventh embodiment of a system for modifying the fluid environment of an ear, the system being constructed according to the present invention and represented generally by reference numeral 711. (For simplicity and clarity, certain components of system 711 that are not critical to the understanding of the present invention are either not shown or described herein or are shown and/or described herein in a simplified manner.)

System 711, which may be designed to be a self-contained unit removably mounted in the ear of a human or other subject, may be similar in most respects to system 11, the primary difference between the two systems being that system 711 may additionally comprise a relief valve 713. Relief valve 713 may be in communication with the ear canal at the distal end of system 711, allowing it to sense pressure increases. When the pressure in the ear canal rises above a certain threshold, relief valve 713 may open, allowing the built-up pressure to be safely released to the ambient environment.

It is to be understood that system 711 may additionally include one or more of the ports (and associated tubing) of system 511 and/or system 611. Also, it is to be understood that relief valves similar or identical to relief valve 713 may be positioned within any one or more of the ports described herein or other similar types of ports.

Also, it is to be understood that system 711 may be modified by positioning the electronics housing outside of the ear, with the relief valve being incorporated into earpiece 13.

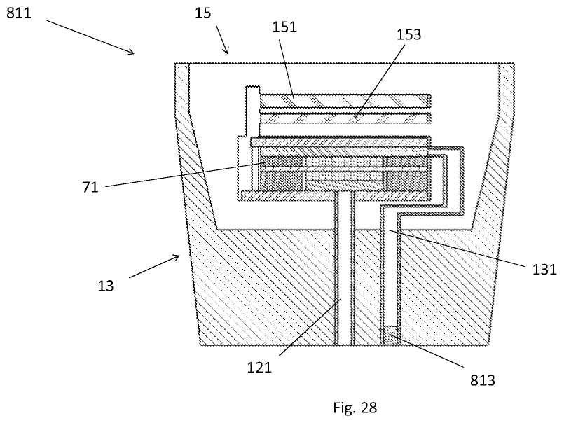

Referring now to FIG. 28, there is shown a section view of an eighth embodiment of a system for modifying the fluid environment of an ear, the system being constructed according to the present invention and represented generally by reference numeral 811. (For simplicity and clarity, certain components of system 811 that are not critical to the understanding of the present invention are either not shown or described herein or are shown and/or described herein in a simplified manner.)

System 811, which may be designed to be a self-contained unit removably mounted in the ear of a human or other subject, may be similar in most respects to system 11, the primary difference between the two systems being that system 811 may additionally comprise a desiccant 813 in-line with first fluid removal tube 131. Desiccant 813 may be a desiccant-infused silicone or other suitable drying material and may be removable and replaceable.

It is to be understood that system 811 may additionally include one or more of the features of system 511, system 611 and/or system 711. Also, it is to be understood that desiccant 813 may be similarly disposed within the fluid removal lumen 225 of system 211.

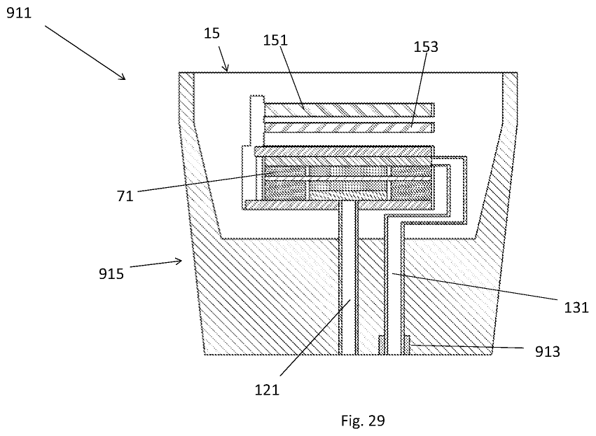

Referring now to FIG. 29, there is shown a section view of a ninth embodiment of a system for modifying the fluid environment of an ear, the system being constructed according to the present invention and represented generally by reference numeral 911. (For simplicity and clarity, certain components of system 911 that are not critical to the understanding of the present invention are either not shown or described herein or are shown and/or described herein in a simplified manner.)

System 911, which may be designed to be a self-contained unit removably mounted in the ear of a human or other subject, may be similar in most respects to system 11, the primary differences between the two systems being that system 911 may additionally comprise an annular desiccant 913 positioned around the distal end of first fluid removal tube 131 and that system 911 may include, instead of earpiece 13, an earpiece 915 having a recess for receiving annular desiccant 913. Desiccant 913 may be a desiccant-infused silicone or other suitable drying material and may be removable and replaceable.

It is to be understood that system 911 may additionally include one or more of the features of system 511, system 611 and/or system 711. Also, it is to be understood that system 911 may be modified along the lines of system 211 so that the electronics housing is placed outside of the ear.

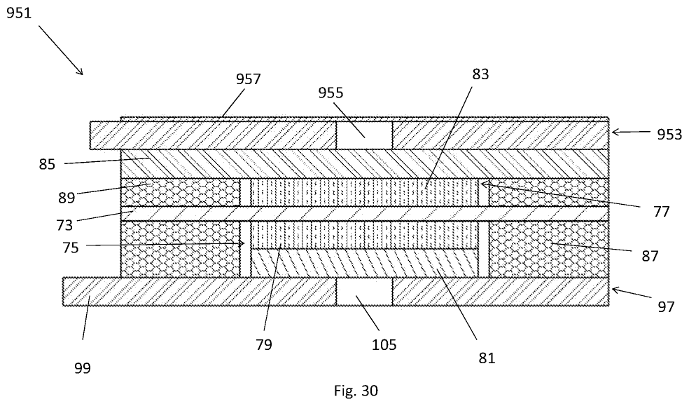

Referring now to FIG. 30, there is shown a section view of an alternative embodiment of an electrochemical gas generator, the electrochemical gas generator being constructed according to the present invention and being represented generally by reference numeral 951. (For simplicity and clarity, certain components of electrochemical gas generator 951 that are not critical to the understanding of the present invention are either not shown or described herein or are shown and/or described herein in a simplified manner.)

Electrochemical gas generator 951 may be similar in many respects to electrochemical gas generator 71. One difference between the two electrochemical gas generators may be that electrochemical gas generator 951 may comprise a current collector 953, instead of current collector 107. Current collector 953 may differ from current collector 107 in that current collector 953 may additionally comprise a through hole 955. Another difference between electrochemical gas generator 951 and electrochemical gas generator 71 may be that electrochemical gas generator 951 may further comprise a vapor transport membrane 957, which may be positioned directly on top of current collector 953.

Through hole 955 may allow water vapor present in the fluid exiting the ear to pass from vapor transport membrane 957 to cathode 77, where it may be used as a reactant. Alternatively, vapor transport membrane 957 may have access to the humidity of ambient air where the humidity may be used as a reactant. Additionally, while vapor transport membrane 957 may allow water vapor to pass, it may prevent oxygen in the exiting gas from mixing with hydrogen created at cathode 77 during electrolysis.

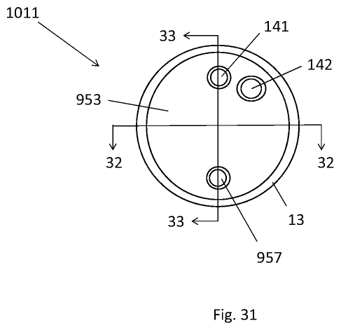

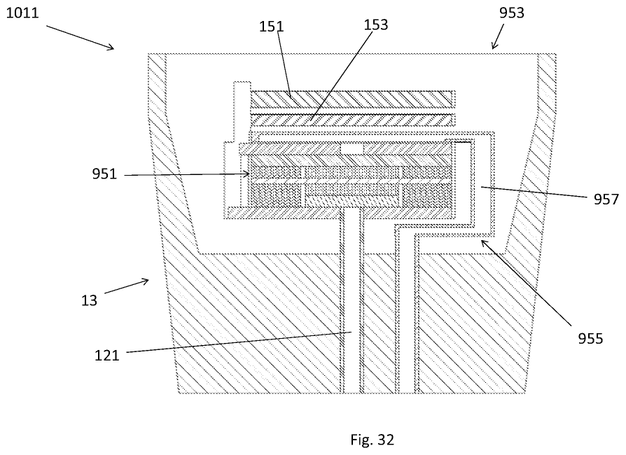

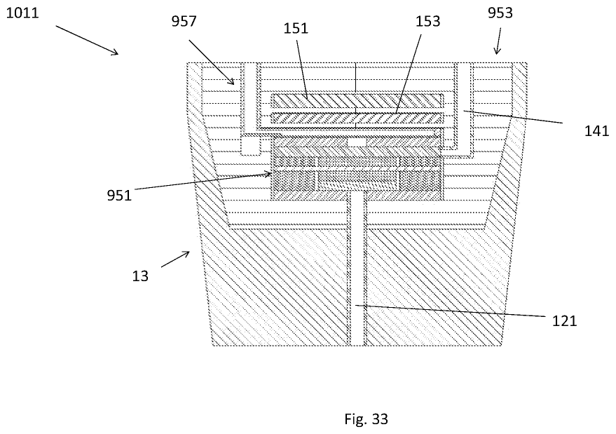

Referring now to FIGS. 31 through 33, there are shown various views of a tenth embodiment of a system for modifying the fluid environment of an ear, the system being constructed according to the present invention and represented generally by reference numeral 1011. (For simplicity and clarity, certain components of system 1011 that are not critical to the understanding of the present invention are either not shown or described herein or are shown and/or described herein in a simplified manner.)

System 1011 may be similar in many respects to system 11. One difference between the two systems may be that, whereas system 11 may include electrochemical gas generator 71, system 1011 may include electrochemical gas generator 951. Another difference between the two systems may be that, whereas system 11 may include electronics housing 15, system 1011 may include electronics housing 953. Electronics housing 953 may be generally similar to electronics housing 15, one difference between the two electronics housings being that electronics housing 953 may be dimensioned to accommodate electrochemical gas generator 951, instead of electrochemical gas generator 71. Another difference between the two electronics housings may be that electronics housing 953 may be shaped to include a fluid removal lumen 955, instead of cavity 61. Fluid removal lumen 955 may extend all the way from the distal end of electronics housing 953 to the proximal end of electronics housing 953, albeit not in a straight line manner.

System 1011 may further comprise a fluid removal tubing 957 positioned in fluid removal lumen 955. Fluid removal tubing 957 may extend from the distal end of earpiece 13 to a location positioned directly over electrochemical gas generator 951 to the proximal end of electronics housing 953. As a result, fluid removal tubing 957 may define a continuous fluid egress path that extends from the ear canal to the ambient environment outside the ear. This continuous fluid egress path may be positioned such that it runs adjacent to vapor transport membrane 957 to allow water vapor from the egress fluid to pass through vapor transport membrane 957 to cathode 77.

It is to be understood that fluid removal tubing 957 may be omitted from system 1101, in which case humid fluid from the ear canal may exit directly through fluid removal lumen 955.

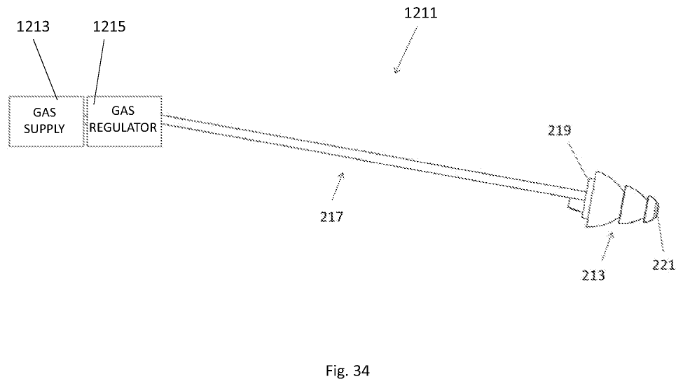

Referring now to FIG. 34, there is shown a partly schematic side view of an eleventh embodiment of a system for modifying the fluid environment of an ear, the system being constructed according to the present invention and represented generally by reference numeral 1211. (For simplicity and clarity, certain components of system 1211 that are not critical to the understanding of the present invention are either not shown or described herein or are shown and/or described herein in a simplified manner.)

System 1211 may be similar in many respects to system 211. One difference between the two systems may be that, whereas system 211 may comprise an electronics housing 215 and an electrochemical gas generator 263, system 1211 may instead comprise a gas supply 1213, which may be in the form of a container holding a predetermined quantity of one or more gases (e.g., a gas cylinder). Alternative gas supplies may include, for example, a pressure swing absorption device, a chemical oxygen release device, or the like, such as are disclosed in U.S. Pat. No. 9,357,764 B2, inventors Tempelman et al., issued Jun. 7, 2016, which is incorporated herein by reference. Gas supply 1213 may be coupled to tubing 217 via a gas regulator 1215 to control the flow of gas from gas supply 1213 to earpiece 213.

System 1211 may be used similarly to system 211.

It is to be understood that features of the various systems disclosed herein may be combined in ways not expressly disclosed.

The present invention may be used in a variety of different situations. For example, and without limitation, some illustrative applications for oxygenation of the ear using the present invention include, but are not limited to, the following: 1) improvement of post-surgical outer ear wound healing; 2) treatment of sudden hearing loss; 3) anaerobic bacteria mitigation and prevention of biofilms produced by facultative bacteria; 4) prevention of otitis externa, or swimmer's ear; and 5) maintaining appropriate relative humidity in the ear canal, specifically in conjunction with hearing aid use. These different applications are discussed in greater detail below.

Oxygenation in the ear using the present invention may improve wound healing following various types of ear surgeries including, but not limited to, tympanoplasty, tympanomastoidectomy, and myringotomy. Electrochemical gas generators, particularly electrochemical oxygen generators, have been used to generate oxygen in situ at skin wounds to improve the healing process for severe burns, diabetic ulcers, and other dermal wounds. Special bandages with electrochemical oxygen generators can provide a means of protecting a dermal wound from the ambient environment while also oxygenating the wound. Since these oxygen-delivering bandages are typically used for difficult to close skin wounds, such as diabetic ulcers, their design has focused on relatively flat surfaces on the external dermal portion of the body. Consequently, prior to the present invention, electrochemical oxygen generators have not been used in connection with wound healing within an ear.

For post-surgical wound healing using the present invention, the system could be worn continuously for the length of time required to achieve healing, which is approximately 12 weeks for the current standard of care of packing the ears and using antibiotic drops but is expected to be shorter with the use of the present system.

Oxygenation in the ear using the present invention may also be useful in the treatment of sudden hearing loss (SHL). The most common treatment for SHL is corticosteroids; however, Hyperbaric Oxygen (HBO) Therapy has been studied recently as a treatment, due to its ability to oxygenate the ear canal. Unfortunately, HBO treatment is logistically difficult, as most hospitals do not have hyperbaric chambers—in the US there are more than six thousand hospitals, but only 190 hyperbaric medicine facilities accredited by the Undersea & Hyperbaric Medical Society. Patients must travel multiple times a week for several weeks to a facility with a chamber, an inconvenience for the typical patient population of older, working adults, and receive at least 1200 minutes, or 20 hours, of treatment. Additionally, HBO therapy is generally not covered by insurance and costs multiple thousands of dollars. A simple, low-cost device that creates an oxygen-rich environment within the ear canal without the logistical issues posed by HBO therapy would allow for wider use of oxygen therapy. As best understood, there is no portable oxygen delivery device suitable for convenient oxygenation of the auditory canal for the treatment of sudden hearing loss.

For the treatment of sudden hearing loss using the present invention, the system could either be worn continuously until hearing returns or it could be worn on a similar schedule as hyperbaric oxygen therapy, 60 to 90 minutes each day for four to two weeks, respectively.

Oxygenation in the ear using the present invention may also be useful in anaerobic bacteria mitigation and prevention of biofilms produced by facultative bacteria. Research has suggested that one cause of acute and chronic otitis media (OM, middle ear infection) may be anaerobic bacteria within the ear. Clinicians have had difficulties confirming this hypothesis due to the practically difficulties of confirming anaerobic infections. Standard diagnostic bacterial culture methods routinely expose the sample to oxygen, thereby killing the anaerobic bacteria. However, oxygen exposure, with its ability to kill anaerobic bacteria, might hold the key to treating acute and chronic OM with fewer antibiotics, as one study has posited that tympanostomy tubes might have the added benefit of allowing ambient oxygen into the middle ear. In the developing world, Chronic Otitis Media continues to be a major cause of preventable hearing loss. To our knowledge, no one has attempted to create an oxygen delivery device suitable for oxygenation of the auditory canal and middle ear for the mitigation of bacteria and prevention of biofilm production.

For bacteria mitigation and prevention of biofilms using the present invention, the system could be worn continuously or intermittently depending on the patient's preference and the extent of bacteria colonization. For Otitis Media (OM) a device designed for safe delivery of appropriate flow rates to the middle ear may resolve some cases of acute or chronic OM with or without treatment with antibiotics before the patient's hearing is damaged. The principle is moderation of the ear microbiome to prevent or resolve difficult to treat anaerobic infections. A simple, low-cost device would allow the treatment to be used in the developing world, where hearing loss from OM is relatively common. The system could be worn preventatively in patients with frequent, recurrent infections or as a treatment for several weeks or more.

Oxygenation in the ear using the present invention may also be useful in the prevention of otitis externa, or swimmer's ear. The direct delivery of gas to the ear canal for the purpose of drying acute humidity is of significance for the prevention of otitis externa, more commonly known as swimmer's ear, an indication responsible for more than 2 million doctor's visits annually in the US. Many national health services, as well as physicians, recommend drying the ear after swimming or showering as a method of preventing otitis externa, with some recommending using a hair dryer on the lowest setting. Based on these recommendations, an effective drying device, designed specifically for drying the ear canal, may prevent otitis externa, if used prophylactically, and may shorten recovery times after diagnosis of otitis externa. Previous approaches that addressed this need have either focused on adapting existing consumer products such as hair dryers for gas delivery to the ear canal or creating a smaller handheld forced air device, with either motorized air delivery or manual air delivery. Adapters for hair dryers reduce the air flow rate and provide a nozzle for easier direction of air into the ear canal. However, even with the adapters, the hair dryers are typically unwieldy and loud, resulting in low consumer uptake. Although smaller handheld devices designed specifically for forced air delivery to the ear canal are quieter and easier to use, they are still indiscreet and limited to intermittent rather than continuous use. Additionally, all three options use ambient air, which could contain contaminants that are unsuitable for the auditory canal, particularly when large quantities of air are directed into the ear. Modified consumer devices and manual air delivery devices do not alert the user to adequate humidity levels, leading to either inadequate or excessive humidity in the ear. To our knowledge, no one has attempted to create a hands-free pure-oxygen delivery device suitable for continuous oxygenation of the auditory canal for the prevention of otitis externa.

For Otitis Externa prevention using the present invention, the system would be worn continuously after activities such as swimming and showering until the humidity alarm alerted the user to optimal humidity in the ear.

Oxygenation of the ear using the present invention may also be useful in the management of chronic humidity for hearing aid and in-the-ear headphone users. During one field study, hearing aid users were separated into two groups based on a self-reported questionnaire. Those who indicated that they noticed a great deal of wax accumulation in their ears and moisture in the canal part of their In-The-Canal hearing device were designated as the “Receiver Problem Group” (RPG), and those who did not were designated as the “No Receiver Problem Group” (NRPG). The RPG group reported their hearing aid was less effective; the NRPG group reported higher satisfaction with their hearing aid. The study showed those in the RPG typically had a measured relative humidity higher than 60%, while no one in the NRPG had a relative humidity above 60%. Those in the RPG did not have a history of heavy earwax accumulation but were observed to have a persistent film of wax on their receiver. By integrating an ELX into a hearing aid or in-the-ear headphone or as an auxiliary to these, the dry oxygen stream would reduce the relative humidity in the ear, decreasing fouling and increasing the efficacy of hearing aids and headphones. The electrolyzer mechanism system provides the lowest humidity oxygen stream. To our knowledge, no one has attempted to integrate a dry oxygen stream with an earpiece to prevent chronic humidity in the ear canal for hearing aid and in-the-ear headphone users.

For chronic humidity management using the present invention, the system would be permanently integrated into the hearing aid or in-the-ear headphone. However, the system would not necessarily operate continuously, but rather, it would be turned on and off by humidity sensor and control electronics so as to maintain the humidity in the ear between 30 and 60% relative humidity. In another use case, the system would provide a minimum flow at all times, increase the flow rate as necessary to reduce the relative humidity in the ear to an acceptable range.

In short, the present inventors have identified several shortcomings of the previous approaches to the diseases or conditions described in the preceding paragraphs that the present invention would remedy. These include the following: (1) Incompatibility with wound surfaces that are not flat, such as the auditory canal; (2) Lack of oxygen for ear surgical wounds and for infected ears; (3) Costly treatments that are not covered by insurance and which must take place at a specialized medical site; (4) Inability to oxygenate the middle ear; (5) Prolonged exposure to loud rotors, motors, or blowers, having an antagonistic acoustic impact; (6) Unwieldy devices that must be actively held in place for the duration of the treatment and which are indiscreet to use; (7) Possible contaminants from the ambient air; (8) Inadequate or excessive drying of the ear leading to negative sequelae; and (9) Inability to maintain a satisfactory humidity within an auditory canal containing an ear piece, either hearing aids or headphones.

The electrochemical gas generator of the present invention is particularly amenable to oxygen delivery to the ear canal. In this scenario, it may be important to have an oxygen delivery rate that is application specific, to mitigate the possible effects of over-drying the ear canal, as well as reduce acoustic impact caused by excess flow. The invention may also include the proper routing of gas streams from the ambient air, to and from the anode and cathode of the electrochemical oxygen generator, and to and from the ear canal and other portions of the ear. Proper routing may provide optimal use of the gas streams as reactants and as the treatment for ear conditions. Optimal use may include provision of optimal pO2, humidity, sterility, and energy usage. The control electronics in the present system may precisely set the current of the electrochemical oxygen generator based on the application's specific flow rate. It shall be readily appreciated that the principles taught in the present application are equally applicable to an ear oxygenation device wherein the electrochemical oxygen generator, itself, is not located near the ear, but rather in another discreet location, such as a hat, headband, neck wrap, pendant, or in a bag. The ear oxygenation device or portions thereof may be disposable after a certain period of time or after use by one patient. Different portions may be suitable for use for different periods of time. The ear oxygen device or portions thereof may be reusable and may be sterilizable or re-sterilizable.

The use of an electrochemical oxygen generator is preferred to other oxygen delivery options, such as adapters for commercial hair dryers, air blowers sized for ear applications, hyperbaric oxygen chambers, and oxygen bottles, due to their settable flow rates, pure oxygen streams, portability and lack of location specificity, ability to be adapted for use in the middle ear, silent operation, reliability, hands-free operation, and ability to alert the user when an optimal humidity has been reached.

Based on the application, the length of time that the system would be used may vary, as may the flow rate.

For simplicity sake, the present system is represented in all figures in a right ear configuration for illustrative purposes, however it is to be understood that all embodiments described below also apply to a left ear configuration.

Additional objects, features, and advantages of the invention are set forth below.

It is an object of the present invention to provide a novel method and system for modifying the fluid environment of an ear, such as by providing a therapeutic gas (e.g., oxygen gas and/or hydrogen gas) to the ear and/or drying the ear (e.g., outer ear and/or middle ear).

It is another object of the present invention to provide a method and system as described above that addresses at least some of the shortcomings associated with existing methods and systems for modifying the fluid environment of the ear.

The above-described system may sometimes be referred to herein as an Ear Oxygenation Device (EOD).

It is still another object of the present invention to provide an Ear Oxygenation Device as described above that is compact, that has a minimal number of parts, that is inexpensive to manufacture, that is electrically efficient, this is reliable, and that is easy to operate. Preferably, the device is designed to be compatible with insertion into the ear canal, is simple and low cost, is quiet, is hands-free, is portable and location independent, is comfortable, and is discreet.

Therefore, according to one embodiment of the invention, there is provided an Ear Oxygenation Device (EOD), the EOD comprising (a) an electrochemical oxygen generator (EOG); (b) control electronics for controlling the EOG's operation; (c) a power source coupled to the EOG and the control electronics for controlling the EOG's operation; (d) means for directing a stream containing an electrochemically generated gas into an ear canal; (e) means for directing gas from out of the ear canal; and (f) one or more housing components comprising some or all of the aforementioned components. An earpiece may be regarded as any portion of the apparatus that is within the ear canal and may include a portion within the middle ear.

In a more detailed feature of the invention, the device may be designed for providing oxygen gas to and/or drying of the right ear.

In a more detailed feature of the invention, the device may be designed for providing oxygen gas to and/or drying of the left ear.

In a more detailed feature of the invention, the device may comprise an electronics housing, and the electronics housing may be designed to fit behind the ear.

In a more detailed feature of the invention, the device may comprise an electronics housing, and the electronics housing may be connected to a delivery port inserted into the ear canal via a tube.

In a more detailed feature of the invention, the device may comprise an electronics housing, and the electronics housing may be detachably coupled to a sterilized, disposable delivery port inserted into the ear canal via a disposable tube set.

In a more detailed feature of the invention, the device may comprise an electronics housing, and the electronics housing may be designed to fit in the ear.

In a more detailed feature of the invention, the device may comprise an electronics housing, and the electronics housing may be incorporated with the delivery port.

In a more detailed feature of the invention, the control electronics may comprise an on/off switch that may be used to control when the device operates.

In a more detailed feature of the invention, the control electronics may include a simple circuit that begins operation when the power source is installed and ends operation when the power source runs out of power and/or is removed from the device.

In a more detailed feature of the invention, the control electronics may include a circuit that provides a constant current to the EOG.

In a more detailed feature of the invention, the control electronics may include a circuit that provides a constant voltage that is converted to a current and provided to the EOG.

In a more detailed feature of the invention, the control electronics may include circuitry that decreases applied current to the EOG and, hence, oxygen production when the power source reaches a low level in order to extend oxygen production life.

In a more detailed feature of the invention, the control electronics may incorporate power monitoring circuitry and a low battery alarm that may provide an audible, visual or motion signal to the user, caregiver or physician.

In a more detailed feature of the invention, the control electronics may interface with one or more sensors including, but not limited to, pressure sensors, humidity sensors, voltage sensors, gas sensors, flow sensors, and accelerometers. The control electronics may use sensors to provide feedback control to control some aspect of the operation of the EOD. These aspects may include on/off or current level.

In a more detailed feature of the invention, the EOD may have a switch that allows a physician to set one of several preprogrammed flow rates to adjust the device based on a desired flow rate dependent upon a patient's ear condition or body size. The control electronics may include an electronic mechanism to provide the current set points for such flow rates.

In a more detailed feature of the invention, the control electronics may include a microprocessor.

In a more detailed feature of the invention, the control electronics may include analog electronics without the use of a microprocessor.

In a more detailed feature of the invention, the control electronics may provide a higher start-up current for a period of time to flush a tubing system and/or the ear canal.

In a more detailed feature of the invention, the control electronics may provide for intermittent provision of oxygen to meet a therapeutic need or to conserve energy.

In a more detailed feature of the invention, the device may be powered by a disposable battery.

In a more detailed feature of the invention, the device housing may have a mechanism for accessing the battery for replacement.

In a more detailed feature of the invention, the device may be powered by a rechargeable battery.

In a more detailed feature of the invention, the device housing may include a mechanism for recharging the battery.

In a more detailed feature of the invention, the device may include an earpiece that extends only partly into the ear canal.

In a more detailed feature of the invention, the device may include an earpiece that extends through the ear canal, terminating near the tympanic membrane.

In a more detailed feature of the invention, the device may include an earpiece that extends through the ear canal and passes through the tympanic membrane via a surgical incision for delivery of oxygen to the middle ear.

In a more detailed feature of the invention, the device may include an earpiece that is extensible to allow for comfortable use for delivery of oxygen to the middle ear.

In a more detailed feature of the invention, the device may include an earpiece that has an oxygen ingress port for oxygen delivery to the ear. The oxygen ingress port path may connect the device's oxygen production electrode to the oxygen delivery location.

In a more detailed feature of the invention, the oxygen ingress port path may be designed for direct oxygen delivery to the ear.

In a more detailed feature of the invention, the oxygen ingress port path may be designed to interact with an air ingress port from outside the ear to provide an oxygen-enriched gas stream that is not pure oxygen. The interaction may include drawing air in via a venturi effect or other convective or diffusive means.

In a more detailed feature of the invention, the oxygen ingress port path may be designed for oxygen to be delivered to the ear in a vortex.

In a more detailed feature of the invention, the oxygen ingress port path may be designed for laminar flow oxygen delivery to the ear.

In a more detailed feature of the invention, the oxygen ingress port path is designed for turbulent flow oxygen delivery to the ear.

In a more detailed feature of the invention, the device may include an earpiece that has a gas egress port for gas release from the ear. The gas egress port path may connect the oxygen delivery location to the ambient environment.

In a more detailed feature of the invention, the oxygen ingress path and the gas egress path may be the same length.

In a more detailed feature of the invention, the oxygen ingress path and the gas egress path may be different lengths.

In a more detailed feature of the invention, one or both of the oxygen ingress path and the gas egress path may terminate at the end of the device earpiece.

In a more detailed feature of the invention, one or both of the oxygen ingress path and the gas egress path may terminate beyond the end of the device earpiece.

In a more detailed feature of the invention, the flow path of the gas egress port through the device earpiece may pass through a cathode support as a means of reactant delivery to the electrochemically-active components.

In a more detailed feature of the invention, the flow path of the gas egress port through the device earpiece may pass adjacent to a vapor transport membrane (VTM). The VTM may separate the hydrogen produced by electrolysis from the oxygen in the gas egress port, allowing the carried vapor to migrate across the membrane to be used as a reactant at the cathode.

In a more detailed feature of the invention, the flow path of the gas egress port may comprise a blower to assist in removal of gas from the ear canal.

In a more detailed feature of the invention, the flow path of the gas egress port may comprise a flap to create convective flow. The flap may be activated by normal head and jaw movement.

In a more detailed feature of the invention, the flow path of the gas egress port may comprise a desiccant to prevent condensate build-up.

In a more detailed feature of the invention, the desiccant may be adjacent to the gas egress port path.

In a more detailed feature of the invention, the desiccant may be replaceable by the user. In a more detailed feature of the invention, the device earpiece may have a pressure relief valve on the gas egress path for emergency pressure release from the ear.

In a more detailed feature of the invention, the electrochemical oxygen generator may be a self-regulating electrochemical gas generator with intrinsic pressure relief according to U.S. Pat. No. 10,557,691, inventors Stone et al., issued Feb. 11, 2020, which is incorporated herein by reference.

In a more detailed feature of the invention, the device earpiece may have a medication delivery port allowing medicine to be delivered to the ear canal without removing the earpiece.

In a more detailed feature of the invention, the device earpiece may have a scope port allowing medical professionals to examine the ear canal without removing the earpiece.

In a more detailed feature of the invention, the device earpiece may have an instrument port allowing medical professionals to perform surgical revisions in the ear canal without removing the earpiece.

In a more detailed feature of the invention, the device earpiece may have a condensate drop out port for removing built-up liquid from the earpiece.

In a more detailed feature of the invention, the various port paths may be a void in the earpiece.

In a more detailed feature of the invention, the various port paths may comprise a tube integrated in the earpiece.

In a more detailed feature of the invention, the quantity of oxygen and its flow rate may be defined by the current set point of the electrochemical oxygen generator and can be varied depending on the application.

In a more detailed feature of the invention, the electrochemical oxygen generator may be a water electrolyzer.

In a more detailed feature of the invention, the electrochemical oxygen generator may be an electrochemical oxygen concentrator.

In a more detailed feature of the invention, the control electronics may include a relative humidity sensor that detects when an optimal humidity has been reached.

In a more detailed feature of the invention, a relative humidity sensor reading may activate an alarm to indicate that optimal humidity has been reached and that the device can be removed from the ear.

In a more detailed feature of the invention, a relative humidity sensor reading may shut down the device when an optimal humidity has been reached and may restart the device when the relative humidity is outside the optimal range.

In a more detailed feature of the invention, the control electronics may include an electrochemical cell voltage sensor.

In a more detailed feature of the invention, the voltage sensor, when operating in an electrolyzer, may detect when the voltage is rising, indicative of nearly dry conditions in the ear.

In a more detailed feature of the invention, a voltage sensor reading may activate an alarm to indicate that optimal humidity has been reached and that the device can be removed from the ear.

In a more detailed feature of the invention, a voltage sensor reading may shut down the device when an optimal humidity has been reached and may restart the device when the relative humidity is outside the optimal range.

In a more detailed feature of the invention, the earpiece may include contours or baffles that effect the gas flow patterns in the ear.

According to another aspect of the invention, the EOD may be used to modify the fluid environment of an ear.

According to another aspect of the invention, the current of the EOD and the resulting oxygen flow rate may be specifically adjusted to modify the fluid environment of the ear to a certain oxygen or humidity level.

The following prophetic example is given for illustrative purposes only and is not meant to be a limitation on the invention described herein or on the claims appended hereto.

### Prophetic Example

An ear oxygenation device similar to that shown in FIG. 11 will be provided, with an earpiece discrete from the housing for the electrochemical oxygen generator, the power source, and the control electronics (referred to hereafter as electronics housing). The electronics housing will be made of a plastic approved for short term use against the skin (such as acrylonitrile butadiene styrene (ABS) plastic, polyether ether ketone (PEEK), or polysulfone) and will contain an electrochemical oxygen concentrator (similar to that shown in FIG. 8). Medical grade silicone tubing to deliver the oxygen to the ear canal will connect the electronics housing to an earpiece also made of medical grade silicone that holds the tubing in the ear as depicted in FIG. 14. The electronics housing will contain two lithium-air batteries that can run the system for 52+ hours with the system operating at a voltage between 0.75 and 1.2 V and a current of 7 mA. The estimated flow rate of oxygen will be 1.58 standard cubic centimeters per hour (SCCH). Before use, a physician will install the batteries, and the electrochemical cell will automatically begin to operate. The electronics housing exterior will be cleaned by the physician with a solution of 70% ethanol. A sterile packet containing the tubing and earpiece will be opened by the physician and connected to the oxygen port protruding from the electronics housing. The electronics housing will be placed behind the ear, and the earpiece will be placed within the ear of the patient three days after a tympanoplasty surgical procedure. The patient will wear the system the majority of the day and night, only removing to shower daily. Once a week, the patient will replace the tubing and earpiece by removing the entire device from their body and disconnecting the tubing from the electronics oxygen port and the ear. The patient will open a new sterile package with tubing and earpiece, connect the tubing to the oxygen port, and place the electronics housing behind the ear and the earpiece within the ear. The patient will be checked periodically (e.g., weekly) by the physician by looking into the ear and assessing the condition of the ear canal and the surgical site. At some point (e.g., 8 weeks), the physician will examine the patient, decide the patient is healed, and discard the entire system in a biohazard disposal container.

The embodiments of the present invention described above are intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. A system for modification of a fluid environment of an ear, the system comprising:
(a) an earpiece, the earpiece being adapted to be mounted in an ear canal, the earpiece comprising a first fluid delivery path and a first fluid removal path; and
(b) a gas source for supplying a gas;
(c) wherein the gas source is fluidly coupled to the first fluid delivery path of the earpiece, whereby gas emitted from the first fluid delivery path causes fluid in the ear to be removed through the first fluid removal path.

2. The system as claimed in claim 1 wherein the gas source comprises an electrochemical gas generating device, the electrochemical gas generating device comprising an electrochemical gas generator, the electrochemical gas generator comprising a first outlet through which gas produced by the electrochemical gas generator is emitted, and wherein the first outlet of the electrochemical gas generator is fluidly coupled to the first fluid delivery path of the earpiece.

3. The system as claimed in claim 2 further comprising an electronics housing, the electrochemical gas generator being disposed within the electronics housing.

4. The system as claimed in claim 3 wherein the electronics housing is adapted to be mounted in the ear canal.

5. The system as claimed in claim 4 wherein the electronics housing is directly mounted on the earpiece.

6. The system as claimed in claim 5 wherein the electronics housing comprises a first fluid delivery path and a first fluid removal path, the first fluid delivery path of the electronics housing being fluidly coupled at a first end to the first outlet of the electrochemical gas generator and being fluidly coupled at a second end to the first fluid delivery path of the earpiece, the first fluid removal path of the electronics housing being fluidly coupled to the first fluid removal path of the earpiece.

7. The system as claimed in claim 6 further comprising a relief valve positioned within the first fluid removal path of the electronics housing.

8. The system as claimed in claim 3 wherein the electronics housing is adapted to be worn outside the ear.

9. The system as claimed in claim 8 further comprising tubing for use in fluidly connecting the outlet of the electrochemical gas generator to the first fluid delivery path of the earpiece.

10. The system as claimed in claim 2 wherein the electrochemical gas generator comprises a water electrolyzer.

11. The system as claimed in claim 10 wherein the gas emitted through the first outlet comprises oxygen gas.

12. The system as claimed in claim 10 wherein the gas emitted through the first outlet comprises hydrogen gas.

13. The system as claimed in claim 10 wherein the gas emitted through the first outlet comprises a mixture of hydrogen gas and oxygen gas.

14. The system as claimed in claim 2 wherein the electrochemical gas generator comprises an electrochemical oxygen concentrator.

15. The system as claimed in claim 2 wherein the electrochemical gas generator comprises a proton exchange membrane, an anode on one face of the proton exchange membrane, a cathode on an opposing face of the proton exchange membrane, an anode current collector coupled to the anode opposite the proton exchange membrane, and a cathode current collector coupled to the cathode opposite the proton exchange membrane, wherein at least one of the anode current collector and the cathode current collector comprises a through hole.

16. The system as claimed in claim 15 wherein each of the anode current collector and the cathode current collector comprises a through hole and wherein the electrochemical gas generator further comprises a vapor transport membrane coupled to the cathode current collector.

17. The system as claimed in claim 2 wherein the electrochemical gas generating device further comprises a power source and control electronics operatively coupled to the electrochemical gas generator.

18. The system as claimed in claim 17 wherein the control electronics comprises a current controller.

19. The system as claimed in claim 18 wherein the control electronics further comprises an on/off switch.

20. The system as claimed in claim 19 wherein the control electronics further comprises a battery monitor.

21. The system as claimed in claim 19 wherein the control electronics further comprises at least one of a microprocessor, a sensor, and an alarm.

22. The system as claimed in claim 18 wherein the control electronics further comprises a voltage regulator.

23. The system as claimed in claim 18 wherein the control electronics further comprises a current selector switch.

24. The system as claimed in claim 23 wherein the control electronics further comprises a microprocessor, a sensor, and an alarm.

25. The system as claimed in claim 1 wherein the earpiece further comprises a tympanostomy tube suitable for insertion through a tympanic membrane of the ear.

26. The system as claimed in claim 1 further comprising at least one of a medicine delivery tube, a scope tube, and an instrument tube, wherein each of the medicine delivery tube, the scope tube and the instrument tube is insertable into the earpiece.

27. The system as claimed in claim 1 further comprising a condensate drop-out port insertable into the earpiece.

28. The system as claimed in claim 1 further comprising a desiccant proximate to a distal end of the first fluid delivery path.

29. The system as claimed in claim 1 wherein the gas source comprises a container holding a quantity of the gas.

30. The system as claimed in claim 29 further comprising a gas regulator fluidly connected between the gas source and the earpiece.

31. A method for modification of a fluid environment of an ear, the method comprising the steps of:
(a) providing the system of claim 1;
(b) implanting the earpiece in an ear; and
(c) delivering gas from the gas source to the earpiece.

32. The method of claim 31 wherein oxygen gas is emitted from the first fluid delivery path.

33. The method of claim 31 wherein hydrogen gas is emitted from the first fluid delivery path.

34. The method of claim 31 wherein oxygen gas and hydrogen gas are emitted from the first fluid delivery path.
